SEQUENCE LISTING (26 sequences)

SEQ ID NO: 1 - protein, 226 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 226 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 3 - protein, 226 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALKFHYTQKSLSLSPG

SEQ ID NO: 4 - protein, 226 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALKYHYTQKSLSLSPG

SEQ ID NO: 5 - protein, 226 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKYHYTQKSLSLSPG

SEQ ID NO: 6 - protein, 226 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHFHYTQKSLSLSPG

SEQ ID NO: 7 - protein, 226 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALRYHYTQKSLSLSPG

SEQ ID NO: 8 - protein, 226 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALRFHYTQKSLSLSPG

SEQ ID NO: 9 - protein, 226 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALRFHYTQKSLSLSPG

SEQ ID NO: 10 - protein, 365 aa, Homo sapiens
MGVPRPQPWALGLLLFLLPGSLGAESHLSLLYHLTAVSSPAPGTPAFWVSGWLGPQQYLSYNSLRGEAEPCGAWVWENQVSWYWEKETTDLRIKEKLFLEAFKALGGKGPYTLQGLLGCELGPDNTSVPTAKFALNGEEFMNFDLKQGTWGGDWPEALAISQRWQQQDKAANKELTFLLFSCPHRLREHLERGRGNLEWKEPPSMRLKARPSSPGFSVLTCSAFSFYPPELQLRFLRNGLAAGTGQGDFGPNSDGSFHASSSLTVKSGDEHHYCCIVQHAGLAQPLRVELESPAKSSVLVVGIVIGVLLLTAAAVGGALLWRRMRSGLPAPWISLRGDDTGVLLPTPGEAQDADLKDVNVIPATA

SEQ ID NO: 11 - protein, 119 aa, Homo sapiens
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 12 - protein, 365 aa, Macaca fascicularis
MRVPRPQPWALGLLLFLLPGSLGAESHLSLLYHLTAVSSPAPGTPAFWVSGWLGPQQYLSYDSLRGQAEPCGAWVWENQVSWYWEKETTDLRIKEKLFLEAFKALGGKGPYTLQGLLGCELSPDNTSVPTAKFALNGEEFMNFDLKQGTWGGDWPEALAISQRWQQQDKAANKELTFLLFSCPHRLREHLERGRGNLEWKEPPSMRLKARPGNPGFSVLTCSAFSFYPPELQLRFLRNGMAAGTGQGDFGPNSDGSFHASSSLTVKSGDEHHYCCIVQHAGLAQPLRVELETPAKSSVLVVGIVIGVLLLTAAAVGGALLWRRMRSGLPAPWISLRGDDTGSLLPTPGEAQDADSKDINVIPATA

SEQ ID NO: 13 - protein, 119 aa, Macaca fascicularis
MSPSVALAVLALLSLSGLEAIQRTPKIQVYSRHPPENGKPNFLNCYVSGFHPSDIEVDLLKNGEKMGKVEHSDLSFSKDWSFYLLYYTEFTPNEKDEYACRVNHVTLSGPRTVKWDRDM

SEQ ID NO: 14 - protein, 365 aa, Mus musculus
MGMPLPWALSLLLVLLPQTWGSETRPPLMYHLTAVSNPSTGLPSFWATGWLGPQQYLTYNSLRQEADPCGAWMWENQVSWYWEKETTDLKSKEQLFLEALKTLEKILNGTYTLQGLLGCELASDNSSVPTAVFALNGEEFMKFNPRIGNWTGEWPETEIVANLWMKQPDAARKESEFLLNSCPERLLGHLERGRRNLEWKEPPSMRLKARPGNSGSSVLTCAAFSFYPPELKFRFLRNGLASGSGNCSTGPNGDGSFHAWSLLEVKRGDEHHYQCQVEHEGLAQPLTVDLDSSARSSVPVVGIVLGLLLVVVAIAGGVLLWGRMRSGLPAPWLSLSGDDSGDLLPGGNLPPEAEPQGANAFPATS

SEQ ID NO: 15 - protein, 119 aa, Mus musculus
MARSVTLVFLVLVSLTGLYAIQKTPQIQVYSRHPPENGKPNILNCYVTQFHPPHIEIQMLKNGKKIPKVEMSDMSFSKDWSFYILAHTEFTPTETDTYACRVKHASMAEPKTVYWDRDM

SEQ ID NO: 16 - protein, 366 aa, Rattus norvegicus
MGMSQPGVLLSLLLVLLPQTWGAEPRLPLMYHLAAVSDLSTGLPSFWATGWLGAQQYLTYNNLRQEADPCGAWIWENQVSWYWEKETTDLKSKEQLFLEAIRTLENQINGTFTLQGLLGCELAPDNSSLPTAVFALNGEEFMRFNPRTGNWSGEWPETDIVGNLWMKQPEAARKESEFLLTSCPERLLGHLERGRQNLEWKEPPSMRLKARPGNSGSSVLTCAAFSFYPPELKFRFLRNGLASGSGNCSTGPNGDGSFHAWSLLEVKRGDEHHYQCQVEHEGLAQPLTVDLDSPARSSVPVVGIILGLLLVVVAIAGGVLLWNRMRSGLPAPWLSLSGDDSGDLLPGGNLPPEAEPQGVNAFPATS

SEQ ID NO: 17 - protein, 119 aa, Rattus norvegicus
MARSVTVIFLVLVSLAVVLAIQKTPQIQVYSRHPPENGKPNFLNCYVSQFHPPQIEIELLKNGKKIPNIEMSDLSFSKDWSFYILAHTEFTPTETDVYACRVKHVTLKEPKTVTWDRDM

SEQ ID NO: 18 - protein, 226 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVDHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 19 - protein, 226 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVDHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKYHYTQKSLSLSPG

SEQ ID NO: 20 - protein, 226 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVDHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALKFHYTQKSLSLSPG

SEQ ID NO: 21 - protein, 226 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVDHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALKYHYTQKSLSLSPG

SEQ ID NO: 22 - protein, 226 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVDHKDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 23 - protein, 226 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVDHKDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKYHYTQKSLSLSPG

SEQ ID NO: 24 - protein, 222 aa, Homo sapiens
VECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 25 - protein, 223 aa, Homo sapiens
PPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 26 - protein, 227 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

(19) United States
(12) Patent Application Publication
(10) Pub. No.: US 2025/0197504 A1
(43) Pub. Date: Jun. 19, 2025

(54) FC FRAGMENTS THAT BIND FCRN AND METHODS OF USE

(71) Applicant: Paragon Therapeutics, Inc., Waltham, MA (US)

(72) Inventors: Yuqi LIU, Belmont, MA (US); Jason Z. OH, Shrewsbury, MA (US); Hussam H. SHAHEEN, Auburn, NH (US); Rita L. VAZ, Lisbon (PT)

(21) Appl. No.: 19/069,168

(22) Filed: Mar. 3, 2025

Related U.S. Application Data
(63) Continuation of application No. PCT/US2024/021300, filed on Mar. 25, 2024.
(60) Provisional application No. 63/492,170, filed on Mar. 24, 2023.

(51) Int. Cl. C07K 16/28

(52) U.S. Cl. CPC C07K 16/283; C07K 2317/52; C07K 2317/94

(57) ABSTRACT

Described herein are Fc fragment variants that bind neonatal Fc receptor (FcRn) that effectively block IgG from binding to FcRn, and methods of use thereof. In certain aspects, described herein are methods of inhibiting FcRn biological activity. In certain aspects, described herein are pharmaceutical compositions comprising the Fc fragments. In certain aspects, the Fc fragments and methods described herein are used for treatment of a disease or disorder related to an antibody (e.g., an autoimmune disease or an unwanted side effect of a therapeutic antibody).

### CROSS-REFERENCE TO RELATED APPLICATIONS

The present application is a Continuation of International Application No. PCT/US2024/021300, filed Mar. 25, 2024, which claims priority to U.S. Provisional Application No. 63/492,170, filed Mar. 24, 2023, the disclosures of which are hereby incorporated by reference in their entireties.

### INCORPORATION-BY-REFERENCE OF SEQUENCE LISTING

The present application contains a Sequence Listing XML which has been submitted electronically in XML format and is hereby incorporated by reference in its entirety. Said XML copy, created Mar. 2, 2025, is named VRD-018US1_SL.xml, and is 29,310 bytes in size.

### BACKGROUND

Immunoglobulin gamma (IgG) antibodies play a key role in the pathology of many disorders, such as autoimmune diseases, inflammatory diseases, and disorders in which the pathology is characterized by over-expression of IgG antibodies (e.g., hypergammaglobulinemia) (see e.g., Junghans, Immunologic Research 16 (1): 29 (1997)).

The half-life of IgG in the serum is prolonged relative to the serum half-life of other plasma proteins (Roopenian et al, J. Immunology 170:3528 (2003); Junghans and Anderson, Proc. Natl. Acad. Sci. USA 93:5512 (1996)). This long half-life is due, in part, to the binding of the Fc region of IgG to the Fc receptor, FcRn (which generally refers to FcRn/b2m complex as its active form in this application unless otherwise specified). Although FcRn was originally characterized as a neonatal transport receptor for maternal IgG, it also functions in adults to protect IgG from degradation. FcRn binds to pinocytosed IgG and protects the IgG from transport to degradative lysosomes by recycling it back to the extracellular compartment where it is released from FcRn and can resume its biological function. This recycling is facilitated by the pH dependent binding of IgG to FcRn, where the IgG/FcRn interaction is strong at acidic endosomal pH, but is weak to non-existent at extracellular physiological pH. Therefore, at physiological pH the IgG is released from binding to FcRn, and the antibody is recycled, thus prolonging the antibody's half-life.

In certain instances, it is desirable to prevent recycling of IgG, for example in autoimmune or inflammatory diseases. Preventing IgG recycling has previously been achieved through agents that reduce or block the binding of IgG to FcRn or that increase the binding of IgG to FcRn at extracellular physiological pH. One example of such agents are antibodies to FcRn (see e.g., WO2002/43658) that block the binding of IgG. Peptides that bind to and antagonize FcRn function have also been disclosed in the art (see e.g., U.S. Pat. Nos. 6,212,022 and 8,101,186). In addition, IgG molecules comprising variant Fc receptors with enhanced FcRn binding and decreased pH dependent-release have been identified (see e.g., U.S. Pat. No. 8,163,881). These IgG molecules occupy FcRn receptors, making them unavailable to bind and recycle other IgG antibodies. Fc fragments comprising two Fc regions that form a homodimer and that occupy FcRn receptors have also been developed that exhibit enhanced FcRn binding and decreased pH dependent-release (see e.g., U.S. Pat. No. 10,316,073) However, there is a need in the art for additional and/or improved agents that reduce or block FcRn binding to the IgG of intact antibodies for use in the treatment of antibody-mediated disorders caused by such intact antibodies.

### SUMMARY

The present disclosure provides, among other things, an Fc fragment variant that binds neonatal Fc receptor (FcRn) with increased affinity to FcRn at endosomal pH (e.g., pH 6.0) and at physiological pH (e.g., pH 7.4) as compared to a wild-type Fc fragment. As described herein, the present invention is, in part, based on the identification of new sets of mutations in the Fc that are significantly effective in inhibiting IgGs from binding to FcRn. In particular, the Fc fragment variants of the present disclosure are characterized with low IC50 values (e.g., ≤2.5 nM) for blocking IgGs from binding to FcRn and high affinity to FcRn at both endosomal and physiological pHs (e.g., ≤10 nM and ≤300 nM, respectively). This is significant because Fc fragment variants of the present invention can be used at a lower dose and/or less frequent administration to achieve therapeutic effect relative to other Fc fragments. Inventive Fc fragment variants of the present invention promise a more potent treatment of diseases associated with pathogenic IgGs including autoimmune diseases, Myasthenia Gravis, and thyroid eye disease (TED).

In certain aspects, described herein is an isolated Fc fragment that binds neonatal Fc receptor (FcRn) from human, cyno, mouse, or rat, the Fc fragment comprising: a variant of the amino acid sequence set forth in SEQ ID NO: 1, wherein the variant comprises at least one amino acid substitution selected from M428L, H433R and N434Y. In some embodiments, the isolated Fc fragment comprises additional amino acid substitutions at one or more of amino acid positions 252, 254, and 256 of SEQ ID NO: 1. In some embodiments, the additional amino acid substitutions are M252Y, S254T, and/or T256E.

In certain aspects, described herein is an isolated Fc fragment that binds neonatal Fc receptor (FcRn) from human, cyno, mouse, or rat, the Fc fragment comprising: a variant of the amino acid sequence set forth in SEQ ID NO: 1, wherein the variant comprises an amino acid substitution at position 428. In some embodiments, the amino acid substitution is M428L. In some embodiments, the Fc fragment comprises additional amino acid substitutions at one or more of amino acid positions 252, 254, 256, 433, and 434 of SEQ ID NO:1. In some embodiments, the additional amino acid substitutions are M252Y, S254T, T256E, H433K, H433R, N434F and/or N434Y.

In certain aspects, described herein is an isolated Fc fragment that binds neonatal Fc receptor (FcRn) from human, cyno, mouse, or rat, the Fc fragment comprising: a variant of the amino acids sequence set forth in SEQ ID NO: 1, wherein the variant comprises an amino acid substitution H433R. In some embodiments, the additional amino acid substitutions are at one or more of amino acid positions 252, 254, 256, 428, and 434 of SEQ ID NO: 1. In some embodiments, the one or more additional amino acid substitutions are M252Y, S254T, T256E, M428L, N434F and/or N434Y.

In certain aspects, described herein is an isolated Fc fragment that binds neonatal Fc receptor (FcRn) from human, cyno, mouse, or rat, the Fc fragment comprising: a variant of the amino acid sequence set forth in SEQ ID NO: 1, wherein the variant comprises an amino acid substitution N434Y. In some embodiments, the Fc fragment comprises an additional amino acid substitution at one or more of amino acid positions 252, 254, 256, 428, and 433 of SEQ ID NO: 1. In some embodiments, the amino acid substitutions are M252Y, S254T, T256E, M428L, H433K, and/or H433R.

In certain aspects, described herein is an isolated Fc fragment that binds neonatal Fc receptor (FcRn) from human, cyno, mouse, or rat, the Fc fragment comprising: a variant of the amino acid sequence set forth in SEQ ID NO: 1 wherein the variant comprises an amino acid substitution H433K, and additional amino acid substitutions at one or more of amino acid positions 252, 254, 256, 428, and 434. In certain embodiments, the additional amino acid substitutions are M252Y, S254T, T256E, M428L, N434F and/or N434Y.

In some embodiments, the isolated Fc fragment described herein comprises a sequence selected from any one of SEQ ID NOs: 3-9.

The contemplated amino acid substitutions described herein result in a Fc fragment with a lower K_{D} for FcRn at pH 6.0 and a measurable K_{D} for FcRn at pH 7.4, compared to an IgG comprising a wild-type Fc region. The Fc region of a wild-type IgG has a K_{D} of approximately 8×10⁻⁷ M for FcRn at pH 6.0 and no detectable binding to FcRn at pH 7.4. The higher affinity for FcRn at pH 6.0 and/or the ability to remain bound to FcRn at pH 7.4 results in higher occupancy of FcRn by the present Fc fragments, thus reducing the amount of FcRn available to bind an IgG comprising a wild-type Fc region. As a result, an IgG comprising a wild-type Fc region is more susceptible to degradation in the lysosome, effectively reducing its half-life.

In some embodiments, the amino acid substitution results in increased Fc fragment half-life compared to an Fc fragment comprising a wild-type human IgG1 Fc region. In some embodiments, the Fc fragment blocks or reduces naturally occurring recycling of IgG antibodies. In some embodiments, the Fc fragment results in increased catabolism of a pathogenic IgG antibody. In some embodiments, the pathogenic IgG antibody is an antibody that is associated with an autoimmune disease.

In some embodiments, the Fc fragment binds to a FcRn sequence set forth in SEQ ID NO: 10-17 with a K_{D} of less than or equal to about 1×10⁻⁸ M or less than or equal to about 1×10⁻⁹ M, 2×10⁻⁹ M, 3×10⁻⁹ M, 4×10⁻⁹ M, 5×10⁻⁹ M, 6×10⁻⁹M, 7×10⁻⁹ M, 8×10⁻⁹M, or 9×10⁻⁹ M, at pH 6.0 as measured by surface plasmon resonance (SPR). In some embodiments, the Fc fragment binds to a FcRn sequence set forth in SEQ ID NO: 10 or 11 with a K_{D} of less than or equal to about 1×10⁻⁸ M, or less than or equal to about 1×10⁻⁹M, 2×10⁻⁹M, 3×10⁻⁹ M, 4×10⁻⁹ M, 5×10⁻⁹ M, 6×10⁻⁹ M, 7×10⁻⁹ M, 8×10⁻⁹M, or 9×10⁻⁹ M, at pH 6.0 as measured by surface plasmon resonance (SPR). In some embodiments, the Fc fragment binds to a FcRn sequence set forth in SEQ ID NO: 16 or 17 with a K_{D} of less than or equal to about 1×10⁻⁸ M, or less than or equal to about 1×10⁻⁹M, 2×10⁻⁹M, 3×10⁻⁹ M, 4×10⁻⁹M, 5×10⁻⁹ M, 6×10⁻⁹M, 7×10⁻⁹ M, 8×10⁻⁹ M, or 9×10⁻⁹ M, at pH 6.0 as measured by surface plasmon resonance (SPR). In some embodiments, the Fc fragment binds to a FcRn sequence set forth in SEQ ID NO: 10-17 with a K_{D} of less than or equal to about 1×10⁻⁸ M, 2×10⁻⁸ M, 3×10⁻⁸ M, 4×10⁻⁸ M, 5×10⁻⁸ M, 6×10⁻⁸ M, 7×10⁻⁸ M, 8×10⁻⁸ M, or 9×10⁻⁸ M at about pH 6.0, as measured by surface plasmon resonance (SPR). In some embodiments, the Fc fragment binds to a FcRn sequence set forth in SEQ ID NO: 12 or 13 with a K_{D} of less than or equal to about 1×10⁻⁸ M, 2×10⁻⁸ M, 3×10⁻⁸ M, 4×10⁻⁸ M, 5×10⁻⁸ M, 6×10⁻⁸ M, 7×10⁻⁸ M, 8×10⁻⁸ M, or 9×10⁻⁸ M, at pH 6.0 as measured by surface plasmon resonance (SPR). In some embodiments, the Fc fragment binds to a FcRn sequence set forth in SEQ ID NO: 10-17 with a K_{D} of less than or equal to about 1, 2, 3, 4, 5, 6, 7, 8, or 9×10⁻¹⁰ M at about pH 6.0, as measured by surface plasmon resonance (SPR). In some embodiments, the Fc fragment binds to a FcRn sequence set forth in SEQ ID NO: 14 or 15 with a K_{D} of less than or equal to about 1×10⁻¹⁰ M, 2×10⁻¹⁰ M, 3×10⁻¹⁰ M, 4×10⁻¹⁰ M, 5×10⁻¹⁰ M, 6×10⁻¹⁰ M, 7×10⁻¹⁰ M, 8×10⁻¹⁰ M, or 9×10⁻¹⁰ M, at pH 6.0 as measured by surface plasmon resonance (SPR).

In some embodiments, the Fc fragment exhibits a melting temperature greater than 55° C. as measured by Differential Scanning Fluorometry (DSF).

In some embodiments, the human Fc fragment exhibits an aggregation temperature equal to or greater than about 65° C., or greater than about 70° C. as measured by Static light scattering (SLS).

In certain aspects, the isolated human Fc fragments disclosed herein are used in the treatment of a disorder or disease related to an antibody, e.g., an autoimmune disease or a disorder associated with an unwanted side effect from a therapeutic antibody. In some embodiments, the isolated human Fc fragments disclosed herein are used in the treatment of a disease or disorder selected from the group consisting of: generalized myasthenia gravis (gMG) chronic inflammatory demyelinating polyneuropathy, myositis, autoimmune encephalitis, myelin oligodendrocyte glycoprotein antibody disorders (MOG-antibody disorder), membranous nephropathy, lupus nephritis, thyroid eye disease, warm autoimmune hemolytic anemia, hemolytic disease of the fetus and newborn, idiopathic thrombocytopenic purpura, primary Sjogrens Syndrome, systemic lupus erythematosus, rheumatoid arthritis, bullous pemphigoid, pemphigus foliaceus, pemphigus vulgaris, and cutaneous lupus erythematosus.

In some embodiments, the isolated Fc fragments disclosed herein are used in the treatment of generalized myasthenia gravis (gMG). In some embodiments, the isolated Fc fragments disclosed herein are used in the treatment of immune thrombocytopenia (ITP). In some embodiments, the treatment reduces disease severity in a patient and disease severity is assessed by an gMG Disease Severity Outcome Measure.

In certain aspects, described herein is an isolated polynucleotide or set of polynucleotides for expression of the isolated Fc fragment of any of the embodiments disclosed herein. Thus, disclosed herein are isolated polynucleotides or sets of polynucleotides encoding the isolated Fc fragment of any of the embodiments disclosed herein, and optionally, wherein the polynucleotide or set of polynucleotides comprises mRNA or cDNA.

In certain aspects, described herein is a vector or set of vectors for expression of the isolated Fc fragment of any of the embodiments disclosed herein comprising the polynucleotide or set of polynucleotides disclosed herein.

In certain aspects, described herein is a host cell comprising the polynucleotide or set of polynucleotides or the vector or set of vectors of disclosed herein.

In certain aspects, described herein is a method of producing an Fc fragment, the method comprising expressing the Fc fragment within the host cell disclosed herein and isolating the expressed Fc fragment.

In certain aspects, described herein is a pharmaceutical composition comprising the isolated Fc fragment of any one of the embodiments disclosed herein and a pharmaceutically acceptable excipient.

In certain aspects, described herein is a kit comprising the isolated Fc fragment of any one of the embodiments disclosed herein or a pharmaceutical composition disclosed herein and instructions for use.

In certain aspects, described herein is a method for treating or preventing a disorder or disease associated with an antibody, e.g., an autoimmune disease or a disorder associated with an unwanted side effect of a therapeutic antibody in a mammalian subject in need thereof, the method comprising administering to the mammalian subject a therapeutically effective amount the isolated Fc fragment of any one of the embodiments disclosed herein or a pharmaceutical composition disclosed herein. In some embodiments, the disease or disorder is selected from the group consisting of: generalized myasthenia gravis (gMG) chronic inflammatory demyelinating polyneuropathy, myositis, autoimmune encephalitis, myelin oligodendrocyte glycoprotein antibody disorders (MOG-antibody disorder), membranous nephropathy, lupus nephritis, thyroid eye disease, warm autoimmune hemolytic anemia, hemolytic disease of the fetus and newborn, idiopathic thrombocytopenia purpura, primary Sjogrens Syndrome, systemic lupus erythematosus, rheumatoid arthritis, bullous pemphigoid, pemphigus foliaceus, pemphigus vulgaris, and cutaneous lupus erythematosus. In some embodiments, the inflammatory disorder or disease is an autoimmune disease. In some embodiments, the inflammatory disorder or disease is gMG. In some embodiments, the method reduces disease severity in a patient and wherein disease severity is assessed by an gMG Disease Severity Outcome Measure.

In certain aspects, described herein is a method for treating a pathology associated with elevated levels of an IgG in a mammalian subject in need thereof, the method comprising administering to the mammalian subject a therapeutically effective amount the Fc fragment of any one of the embodiments disclosed herein or a pharmaceutical composition disclosed herein.

In certain aspects, described herein is a method of reducing biological activity of an IgG in a mammalian subject in need thereof, the method comprising administering to the mammalian subject a therapeutically effective amount the Fc fragment of any one of the embodiments disclosed herein or a pharmaceutical composition disclosed herein. In certain embodiments, the disease is an autoimmune disease.

In certain aspects, described herein is a method of preventing a disorder in a mammalian subject in need thereof, the method comprising administering to the mammalian subject a therapeutically effective amount the isolated Fc fragment or a pharmaceutical composition described herein, wherein the disorder is an unwanted side-effect of a therapeutic antibody.

In one aspect, the present embodiments provide, among other things, an Fc fragment variant that binds neonatal Fc receptor (FcRn), wherein the Fc fragment variant comprises amino acid substitutions M428L and N434F as compared to an amino acid sequence set forth in SEQ ID NO: 1.

In some embodiments, the Fc fragment variant further comprises an amino acid substitution at position 433 as compared to an amino acid sequence set forth in SEQ ID NO: 1. In some embodiments, the Fc fragment comprises an amino acid substitution of H433K or H433R as compared to an amino acid sequence set forth in SEQ ID NO: 1. In some embodiments, the Fc fragment comprises an amino acid substitution of N434F as compared to an amino acid sequence set forth in SEQ ID NO: 1. In some embodiments, the Fc fragment comprises amino acid substitutions of H433K and N434F as compared to an amino acid sequence set forth in SEQ ID NO: 1. In some embodiments, the comprises amino acid substitutions of H433R and N434F as compared to an amino acid sequence set forth in SEQ ID NO: 1.

In one aspect, the present invention provides, among other things, an Fc fragment variant that binds neonatal Fc receptor (FcRn), wherein the Fc fragment variant comprises amino acid substitutions (i) N434Y and (ii) H433R or H433K as compared to an amino acid sequence set forth in SEQ ID NO: 1.

In some embodiments, Fc fragment comprises amino acid substitutions of H433K and N434Y as compared to an amino acid sequence set forth in SEQ ID NO: 1. In some embodiments, the Fc fragment comprises amino acid substitutions of H433R and N434Y as compared to an amino acid sequence set forth in SEQ ID NO: 1.

In one aspect, the present invention provides, among other things, an Fc fragment variant that binds neonatal Fc receptor (FcRn), wherein the Fc fragment variant comprises amino acid substitutions M428L and N434Y as compared to an amino acid sequence set forth in SEQ ID NO: 1.

In some embodiments, an Fc fragment further comprises an amino acid substitution of H433K.

In one aspect, the present invention provides, among other things, an Fc fragment variant that binds neonatal Fc receptor (FcRn), wherein the Fc fragment variant comprises amino acid substitutions M428L and H433R as compared to an amino acid sequence set forth in SEQ ID NO: 1.

In some embodiments, the Fc fragment further comprises an amino acid substitution of N434Y.

In one aspect, the present invention provides, among other things, an Fc fragment variant that binds neonatal Fc receptor (FcRn), wherein the Fc fragment variant comprises amino acid substitutions H433R and H434F as compared to an amino acid sequence set forth in SEQ ID NO: 1.

In some embodiments, an Fc fragment further comprises amino acid substitutions of M252Y, S254T, and T256E.

In some embodiments, the Fc fragment comprises amino acid substitutions M252Y, S254T, T256E, M428L, H433K, and N434F. In some embodiments, the Fc fragment comprises amino acid substitutions M252Y, S254T, T256E, M428L, H433K, and N434Y. In some embodiments, the Fc fragment comprises amino acid substitutions M252Y, S254T, T256E, H433K, and N434Y. In some embodiments, the Fc fragment comprises amino acid substitutions M252Y, S254T, T256E, M428L, and N434F. In some embodiments, the Fc fragment comprises amino acid substitutions M252Y, S254T, T256E, H433R, and N434F. In some embodiments, the Fc fragment comprises amino acid substitutions M252Y, S254T, T256E, M428L, H433R, and N434F.

In some embodiments, the Fc fragment variant does not comprise an amino substitution of L309D.

In some embodiments, the Fc fragment comprises an amino acid sequence that is at least 80%, 83%, 85%, 88%, 90%, 91%, 92%, 93%, 94%, 95%, 96%, 97%, 98%, and 99% identical to SEQ ID NO: 3. In some embodiments, the Fc fragment comprises an amino acid sequence of SEQ ID NO: 3.

In some embodiments, the Fc fragment comprises an amino acid sequence that is at least 80%, 83%, 85%, 88%, 90%, 91%, 92%, 93%, 94%, 95%, 96%, 97%, 98%, and 99% identical to SEQ ID NO: 4. In some embodiments, the Fc fragment comprises an amino acid sequence of SEQ ID NO: 4.

In some embodiments, the Fc fragment comprises an amino acid sequence that is at least 80%, 83%, 85%, 88%, 90%, 91%, 92%, 93%, 94%, 95%, 96%, 97%, 98%, and 99% identical to SEQ ID NO: 5. In some embodiments, the Fc fragment comprises an amino acid sequence of SEQ ID NO: 5.

In some embodiments, the Fc fragment comprises an amino acid sequence that is at least 80%, 83%, 85%, 88%, 90%, 91%, 92%, 93%, 94%, 95%, 96%, 97%, 98%, and 99% identical to SEQ ID NO: 6. In some embodiments, the Fc fragment comprises an amino acid sequence of SEQ ID NO: 6.

In some embodiments, the Fc fragment comprises an amino acid sequence that is at least 80%, 83%, 85%, 88%, 90%, 91%, 92%, 93%, 94%, 95%, 96%, 97%, 98%, and 99% identical to SEQ ID NO: 7. In some embodiments, the Fc fragment comprises an amino acid sequence of SEQ ID NO: 7.

In some embodiments, the Fc fragment comprises an amino acid sequence that is at least 80%, 83%, 85%, 88%, 90%, 91%, 92%, 93%, 94%, 95%, 96%, 97%, 98%, and 99% identical to SEQ ID NO: 8. In some embodiments, the Fc fragment comprises an amino acid sequence of SEQ ID NO: 8.

In some embodiments, the Fc fragment comprises an amino acid sequence that is at least 80%, 83%, 85%, 88%, 90%, 91%, 92%, 93%, 94%, 95%, 96%, 97%, 98%, and 99% identical to SEQ ID NO: 9. In some embodiments, the Fc fragment comprises an amino acid sequence of SEQ ID NO: 9.

In some embodiments, an Fc fragment variant inhibits an IgG from binding to human FcRn with an IC50 value of less than 2.6 nM. In some embodiments, an Fc fragment variant inhibits an IgG from binding to human FcRn with an IC50 value of less than 2.5 nM. In some embodiments, an Fc fragment variant inhibits an IgG from binding to human FcRn with an IC50 value of less than 2.3 nM. In some embodiments, an Fc fragment variant inhibits an IgG from binding to human FcRn with an IC50 value of less than 2.2 nM. In some embodiments, an Fc fragment variant inhibits an IgG from binding to human FcRn with an IC50 value of less than 2.0 nM. In some embodiments, an Fc fragment variant inhibits an IgG from binding to human FcRn with an IC50 value of less than 1.9 nM. In some embodiments, an Fc fragment variant inhibits an IgG from binding to human FcRn with an IC50 value of less than 1.8 nM. In some embodiments, an Fc fragment variant inhibits an IgG from binding to human FcRn with an IC50 value of less than 1.7 nM. In some embodiments, an Fc fragment variant inhibits an IgG from binding to human FcRn with an IC50 value of less than 1.6 nM. In some embodiments, an Fc fragment variant inhibits an IgG from binding to human FcRn with an IC50 value of less than 1.5 nM. In some embodiments, an Fc fragment variant inhibits an IgG from binding to human FcRn with an IC50 value of less than 1.4 nM. In some embodiments, an Fc fragment variant inhibits an IgG from binding to human FcRn with an IC50 value of less than 1.3 nM. In some embodiments, an Fc fragment variant inhibits an IgG from binding to human FcRn with an IC50 value of less than 1.2 nM. In some embodiments, an Fc fragment variant inhibits an IgG from binding to human FcRn with an IC50 value of less than 1.1 nM. In some embodiments, an Fc fragment variant inhibits an IgG from binding to human FcRn with an IC50 value of less than 1.0 nM. In some embodiments, an Fc fragment variant inhibits an IgG from binding to human FcRn with an IC50 value of less than 0.9 nM. In some embodiments, an Fc fragment variant inhibits an IgG from binding to human FcRn with an IC50 value of less than 0.8 nM.

In some embodiments, an Fc fragment variant inhibits an IgG from binding to human FcRn with an IC50 value of from about 0.5-2.2 nM. In some embodiments, an Fc fragment variant inhibits an IgG from binding to human FcRn with an IC50 value of between 0.7-2.0 nM. In some embodiments, an Fc fragment variant inhibits an IgG from binding to human FcRn with an IC50 value of from about 0.7-1.8 nM. In some embodiments, an Fc fragment variant inhibits an IgG from binding to human FcRn with an IC50 value of from about 0.7-1.6 nM. In some embodiments, an Fc fragment variant inhibits an IgG from binding to human FcRn with an IC50 value of from about 0.7-1.5 nM. In some embodiments, an Fc fragment variant inhibits an IgG from binding to human FcRn with an IC50 value of from about 0.7-1.4 nM. In some embodiments, an Fc fragment variant inhibits an IgG from binding to human FcRn with an IC50 value of from about 0.7-1.3 nM. In some embodiments, an Fc fragment variant inhibits an IgG from binding to human FcRn with an IC50 value of from about 0.7-1.2 nM. In some embodiments, an Fc fragment variant inhibits an IgG from binding to human FcRn with an IC50 value of from about 0.7-1.2 nM. In some embodiments, an Fc fragment variant inhibits an IgG from binding to human FcRn with an IC50 value of from about 0.7-1.0 nM.

In some embodiments, an Fc fragment variant inhibits an IgG from binding to human FcRn with an IC50 value of 2.0 nM. In some embodiments, an Fc fragment variant inhibits an IgG from binding to human FcRn with an IC50 value of 1.9 nM. In some embodiments, an Fc fragment variant inhibits an IgG from binding to human FcRn with an IC50 value of 1.8 nM. In some embodiments, an Fc fragment variant inhibits an IgG from binding to human FcRn with an IC50 value of 1.7 nM. In some embodiments, an Fc fragment variant inhibits an IgG from binding to human FcRn with an IC50 value of 1.5 nM. In some embodiments, an Fc fragment variant inhibits an IgG from binding to human FcRn with an IC50 value of 1.4 nM. In some embodiments, an Fc fragment variant inhibits an IgG from binding to human FcRn with an IC50 value of 1.3 nM. In some embodiments, an Fc fragment variant inhibits an IgG from binding to human FcRn with an IC50 value of 1.2 nM. In some embodiments, an Fc fragment variant inhibits an IgG from binding to human FcRn with an IC50 value of 1.1 nM. In some embodiments, an Fc fragment variant inhibits an IgG from binding to human FcRn with an IC50 value of 1.0 nM. In some embodiments, an Fc fragment variant inhibits an IgG from binding to human FcRn with an IC50 value of 0.9 nM. In some embodiments, an Fc fragment variant inhibits an IgG from binding to human FcRn with an IC50 value of 0.8 nM. In some embodiments, an Fc fragment variant inhibits an IgG from binding to human FcRn with an IC50 value of 0.7 nM. In some embodiments, an Fc fragment variant inhibits an IgG from binding to human FcRn with an IC50 value of 0.6 nM.

In some embodiments, an Fc fragment variant effectively inhibits an IgG from binding to human FcRn at pHs from about 5.8-7.5. In some embodiments, an Fc fragment variant effectively inhibits an IgG from binding to human FcRn at pHs from about 6.0-7.4.

In some embodiments, an Fc fragment variant effectively inhibits an IgG from binding to human FcRn at pH 6.0. In some embodiments, an Fc fragment variant effectively inhibits an IgG from binding to human FcRn at pH 7.0. In some embodiments, an Fc fragment variant effectively inhibits an IgG from binding to human FcRn at pH 7.4.

In some embodiments, an Fc fragment variant inhibits an IgG from binding to human FcRn at pH 6.0 with an IC50 value of from about 0.7-2.2 nM. In some embodiments, an Fc fragment variant inhibits an IgG from binding to human FcRn at pH 7.4 with an IC50 value of from about 0.7-2.2 nM.

In some embodiments, an Fc fragment is fused or complexed to a half-life extension domain. In some embodiments, a half-life extension domain is a protein. In some embodiments, a half-life extension domain is a polypeptide. In some embodiments, a half-life extension domain is a peptide. In some embodiments, a half-life extension domain is an antibody. In some embodiments, a half-life extension domain is an antibody fragment. In some embodiments, a half-life extension domain is an scFv. In some embodiments, a half-life extension domain is a sdAb. In some embodiments, a half-life extension domain is a Fab. In some embodiments, a half-life extension domain is a VHH. In some embodiments, a half-life extension domain is a or variable new antigen receptors (VNAR).

In some embodiments, a half-life extension domain is an albumin. In some embodiments, a half-life extension domain is an albumin-binding domain. In some embodiments, a half-life extension domain is aa HSA binding domain.

In one aspect, the present invention provides, among other things, a method of inhibiting IgG from binding to FcRn by administering an Fc fragment variant described herein.

### DETAILED DESCRIPTION

### Definitions

Unless otherwise defined, all terms of art, notations and other scientific terminology used herein are intended to have the meanings commonly understood by those of skill in the art. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 4th ed. (2012) Cold Spring Harbor Laboratory Press, Cold Spring Harbor, NY. As appropriate, procedures involving the use of commercially available kits and reagents are generally carried out in accordance with manufacturer-defined protocols and conditions unless otherwise noted.

As used herein, the singular form “a”, “an”, and “the” includes plural references unless indicated otherwise.

It is understood that aspects and embodiments of the invention described herein include “comprising,” “consisting,” and “consisting essentially of” aspects and embodiments.

For all compositions described herein, and all methods using a composition described herein, the compositions can either comprise the listed components or steps, or can “consist essentially of” the listed components or steps. When a composition is described as “consisting essentially of” the listed components, the composition contains the components listed, and may contain other components which do not substantially affect the condition being treated, but do not contain any other components which substantially affect the condition being treated other than those components expressly listed; or, if the composition does contain extra components other than those listed which substantially affect the condition being treated, the composition does not contain a sufficient concentration or amount of the extra components to substantially affect the condition being treated. When a method is described as “consisting essentially of” the listed steps, the method contains the steps listed, and may contain other steps that do not substantially affect the condition being treated, but the method does not contain any other steps which substantially affect the condition being treated other than those steps expressly listed. As a non-limiting specific example, when a composition is described as ‘consisting essentially of’ a component, the composition may additionally contain any amount of pharmaceutically acceptable carriers, vehicles, or diluents and other such components which do not substantially affect the condition being treated.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The terms “host cell,” “host cell line,” and “host cell culture” are used interchangeably and refer to cells into which an exogenous nucleic acid has been introduced, and the progeny of such cells. Host cells include “transformants” (or “transformed cells”) and “transfectants” (or “transfected cells”), which each include the primary transformed or transfected cell and progeny derived therefrom. Such progeny may not be completely identical in nucleic acid content to a parent cell, and may contain mutations. A “recombinant host cell” or “host cell” refers to a cell that includes an exogenous polynucleotide, regardless of the method used for insertion, for example, direct uptake, transduction, f-mating, or other methods known in the art to create recombinant host cells.

As used herein, the term “eukaryote” refers to organisms belonging to the phylogenetic domain Eucarya such as animals (including but not limited to, mammals, insects, reptiles, birds, etc.), ciliates, plants (including but not limited to, monocots, dicots, algae, etc.), fungi, yeasts, flagellates, microsporidia, protists, etc.

As used herein, the term “prokaryote” refers to prokaryotic organisms. For example, a non-eukaryotic organism can belong to the Eubacteria (including but not limited to, Escherichia coli, Thermus thermophilus, Bacillus stearothermophilus, Pseudomonas fluorescens, Pseudomonas aeruginosa, Pseudomonas putida, etc.) phylogenetic domain, or the Archaea (including but not limited to, Methanococcus jannaschii, Methanobacterium thermautotrophicum, Halobacterium such as Haloferax volcanii and Halobacterium species NRC-1, Archaeoglobus fulgidus, Pyrococcus furiosus, Pyrococcus horikoshii, Aeropyrum pernix, etc.) phylogenetic domain.

An “effective amount” or “therapeutically effective amount” as used herein refers to an amount of therapeutic compound, such as an Fc fragment, administered to an individual, either as a single dose or as part of a series of doses, which is effective to produce or contribute to a desired therapeutic effect, either alone or in combination with another therapeutic modality. Examples of a desired therapeutic effect is reduction in IgG levels amelioration of one or more symptoms. An effective amount may be given in one or more dosages.

The term “treating” (and variations thereof such as “treat” or “treatment”) refers to clinical intervention in an attempt to alter the natural course of a disease or condition in a subject in need thereof. Treatment can be performed during the course of clinical pathology. Desirable effects of treatment include preventing recurrence of disease, alleviation of symptoms, diminishment of any direct or indirect pathological consequences of the disease, preventing metastasis, decreasing the rate of disease progression, amelioration or palliation of the disease state, and remission or improved prognosis.

The term “sufficient amount” means an amount sufficient to produce a desired effect, e.g., an amount sufficient to modulate an immune response in a subject.

As used herein, the term “subject” or “individual” means a mammalian subject. Exemplary subjects include humans, monkeys, dogs, cats, mice, rats, cows, horses, camels, goats, rabbits, and sheep. In certain embodiments, the subject is a human.

The term “in vitro” refers to processes that occur in a living cell growing separate from a living organism, e.g., growing in tissue culture.

The term “in vivo” refers to processes that occur in a living organism.

The term “package insert” is used to refer to instructions customarily included in commercial packages of therapeutic or diagnostic products (e.g., kits) that contain information about the indications, usage, dosage, administration, combination therapy, contraindications and/or warnings concerning the use of such therapeutic or diagnostic products.

The term “pharmaceutical composition” refers to a preparation which is in such form as to permit the biological activity of an active ingredient contained therein to be effective in treating a subject, and which contains no additional components which are unacceptably toxic to the subject in the amounts provided in the pharmaceutical composition.

The terms “co-administration”, “co-administer”, and “in combination with” include the administration of two or more therapeutic agents either simultaneously, concurrently or sequentially within no specific time limits. In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to the administration of a second therapeutic agent.

The terms “modulate” and “modulation” refer to reducing or inhibiting or, alternatively, activating or increasing, a recited variable.

The terms “increase” and “activate” refer to an increase of 10%, 20%, 30%, 40%, 50%, 60%, 70%, 75%, 80%, 85%, 90%, 95%, 100%, 2-fold, 3-fold, 4-fold, 5-fold, 10-fold, 20-fold, 50-fold, 100-fold, or greater in a recited variable.

The terms “reduce” and “inhibit” refer to a decrease of 10%, 20%, 30%, 40%, 50%, 60%, 70%, 75%, 80%, 85%, 90%, 95%, 2-fold, 3-fold, 4-fold, 5-fold, 10-fold, 20-fold, 50-fold, 100-fold, or greater in a recited variable.

The term “about” indicates and encompasses an indicated value and a range above and below that value. In certain embodiments, the term “about” indicates the designated value ±10%, ±5%, or ±1%. In certain embodiments, where applicable, the term “about” indicates the designated value(s)±one standard deviation of that value(s).

For any of the structural and functional characteristics described herein, methods of determining these characteristics are known in the art.

The term “optionally” is meant, when used sequentially, to include from one to all of the enumerated combinations and contemplates all sub-combinations.

The term “amino acid” refers to the twenty common naturally occurring amino acids. Naturally occurring amino acids include alanine (Ala; A), arginine (Arg; R), asparagine (Asn; N), aspartic acid (Asp; D), cysteine (Cys; C); glutamic acid (Glu; E), glutamine (Gln; Q), Glycine (Gly; G); histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

The term “affinity” refers to the strength of the sum total of non-covalent interactions between a single binding site of a molecule (e.g., an Fc fragment) and its binding partner (e.g., FcRn). Unless indicated otherwise, as used herein, “affinity” refers to intrinsic binding affinity, which reflects a 1:1 interaction between members of a binding pair (e.g., an Fc fragment and FcRn). Affinity is indirectly proportional to K_{D}.

The term “variant”, as used herein when referring to an amino acid sequence, refers to an amino acid sequence comprising one or more changes in the sequence compared to a reference amino acid sequence, including one or more substitutions compared to a reference amino acid sequence.

The term “kd” (sec-1), as used herein, refers to the dissociation rate constant of a particular antibody-antigen or protein-protein interaction. This value is also referred to as the koff value.

The term “ka” (M-1×sec-1), as used herein, refers to the association rate constant of a particular antibody-antigen interaction or protein-protein. This value is also referred to as the kon value.

The term “KD” or “K_{D}”) (M), as used herein, refers to the dissociation equilibrium constant of a particular antibody-antigen or protein-protein interaction. KD=kd/ka. In some embodiments, the affinity of a protein is described in terms of the KD for an interaction between such protein and its binding partner. For clarity, as known in the art, a smaller KD value indicates a higher affinity interaction, while a larger KD value indicates a lower affinity interaction.

The term “measurable KD” or “measurable K_{D}” as used herein, means a KD value that is less than 1M, less than 0.1M, less than 0.01M, less than 0.001M, less than 1×10⁻⁴ M, less than 1×10⁻⁵ M, or less than 1×10⁻⁶ M.

The term “KA” (M-1), as used herein, refers to the association equilibrium constant of a particular antibody-antigen or protein-protein interaction. KA=ka/kd.

The term “antibody” is used herein in its broadest sense and includes certain types of immunoglobulin molecules comprising one or more antigen-binding domains that specifically bind to an antigen or epitope. An antibody specifically includes intact antibodies (e.g., intact immunoglobulins), antibody fragments, and multi-specific antibodies.

The terms “full length antibody,” “intact antibody,” and “whole antibody” are used herein interchangeably to refer to an antibody having a structure substantially similar to a naturally occurring antibody structure and having heavy chains that comprise an Fc region. For example, when used to refer to an IgG molecule, a “full length antibody” is an antibody that comprises two heavy chains and two light chains.

The term “Fc domain” or “Fc region” or “Fc fragment” herein is used to define a C-terminal region of an immunoglobulin heavy chain that contains at least a portion of the constant region. The term includes native sequence Fc regions and variant Fc regions.

A “Fc fragment,” as provided herein, refers to a fragment of the Fc domain which specifically binds to the target protein FcRn. In some embodiments, a Fc fragment is a variant of SEQ ID NO:1.

The term “human Fc fragment” refers to an Fc fragment which possesses an amino acid sequence corresponding to that of an Fc region of an antibody produced by a human or a human cell, or derived from a non-human source that utilizes a human antibody repertoire or human antibody-encoding sequences (e.g., obtained from human sources or designed de novo).

The term “substantially purified” refers to a construct described herein, or variant thereof that may be substantially or essentially free of components that normally accompany or interact with the protein as found in its naturally occurring environment, i.e. a native cell, or host cell in the case of recombinantly produced protein that in certain embodiments, is substantially free of cellular material includes preparations of protein having less than about 30%, less than about 25%, less than about 20%, less than about 15%, less than about 10%, less than about 5%, less than about 4%, less than about 3%, less than about 2%, or less than about 1% (by dry weight) of contaminating protein.

Ranges recited herein are understood to be shorthand for all of the values within the range, inclusive of the recited endpoints. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

It must be noted that, as used in the specification and the appended claims, the singular forms “a,” “an” and “the” include plural referents unless the context clearly dictates otherwise.

### Fc Fragments

### Fc Fragment Structure

Fc fragments can include those described herein such as the amino acid sequences set forth in the tables. In some embodiments, the Fc fragment is an IgG subclass IgG1, IgG2 or IgG4.

In certain embodiments, the Fc fragments are produced by recombinant cells engineered to express the desired constant domains.

### Sequences of FcRn-Binding Fc Fragments

In certain aspects, described herein is an isolated Fc fragment that binds neonatal Fc receptor (FcRn) from human, cyno, mouse, or rat, the Fc fragment comprising: a variant of the amino acid sequence set forth in SEQ ID NO: 1, wherein the variant comprises at least one amino acid substitution selected from M428L, H433R and N434Y. In some embodiments, the isolated Fc fragment comprises additional amino acid substitutions at one or more of amino acid positions 252, 254, and 256 of SEQ ID NO: 1. In some embodiments, the additional amino acid substitutions are M252Y, S254T, and/or T256E.

In certain aspects, described herein is an isolated Fc fragment that binds neonatal Fc receptor (FcRn) from human, cyno, mouse, or rat, the Fc fragment comprising: a variant of the amino acid sequence set forth in SEQ ID NO: 1, wherein the variant comprises an amino acid substitution at position 428. In some embodiments, the amino acid substitution is M428L. In some embodiments, the Fc fragment comprises additional amino acid substitutions at one or more of amino acid positions 252, 254, 256, 433, and 434 of SEQ ID NO:1. In some embodiments, the additional amino acid substitutions are M252Y, S254T, T256E, H433K, H433R, N434F and/or N434Y.

In certain aspects, described herein is an isolated Fc fragment that binds neonatal Fc receptor (FcRn) from human, cyno, mouse, or rat, the Fc fragment comprising: a variant of the amino acids sequence set forth in SEQ ID NO: 1, wherein the variant comprises an amino acid substitution H433R. In some embodiments, the additional amino acid substitutions are at one or more of amino acid positions 252, 254, 256, 428, and 434 of SEQ ID NO: 1. In some embodiments, the one or more additional amino acid substitutions are M252Y, S254T, T256E, M428L, N434F and/or N434Y.

In certain aspects, described herein is an isolated Fc fragment that binds neonatal Fc receptor (FcRn) from human, cyno, mouse, or rat, the Fc fragment comprising: a variant of the amino acid sequence set forth in SEQ ID NO: 1, wherein the variant comprises an amino acid substitution N434Y. In some embodiments, the Fc fragment comprises an additional amino acid substitution at one or more of amino acid positions 252, 254, 256, 428, and 433 of SEQ ID NO: 1. In some embodiments, the amino acid substitutions are M252Y, S254T, T256E, M428L, H433K, and/or H433R.

In certain aspects, described herein is an isolated Fc fragment that binds neonatal Fc receptor (FcRn) from human, cyno, mouse, or rat, the Fc fragment comprising: a variant of the amino acid sequence set forth in SEQ ID NO: 1 wherein the variant comprises an amino acid substitution H433K, and additional amino acid substitutions at one or more of amino acid positions 252, 254, 256, 428, and 434. In certain embodiments, the additional amino acid substitutions are M252Y, S254T, T256E, M428L, N434F and/or N434Y.

In certain embodiments, the Fc fragment comprises a sequence selected from the sequences set forth in SEQ ID NO: 3-9.

In certain embodiments, the Fc fragment comprises a sequence that is at least 85%, 90%, 91%, 92%, 93%, 94%, 95%, 96%, 97%, 98%, or 99% identical to an amino acid sequence comprising SEQ ID NO: 3, provided that such fragment comprises the mutations of M252Y, S254T, T256E, M428L, H433K, and N434F.

In some embodiments, an Fc fragment variant comprises an amino acid sequence that is at least 80% identical to SEQ ID NO: 3. In some embodiments, an Fc fragment variant comprises an amino acid sequence that is at least 85% identical to SEQ ID NO: 3. In some embodiments, an Fc fragment variant comprises an amino acid sequence that is at least 88% identical to SEQ ID NO: 3. In some embodiments, an Fc fragment variant comprises an amino acid sequence that is at least 90% identical to SEQ ID NO: 3. In some embodiments, an Fc fragment variant comprises an amino acid sequence that is at least 91% identical to SEQ ID NO: 3. In some embodiments, an Fc fragment variant comprises an amino acid sequence that is at least 92% identical to SEQ ID NO: 3. In some embodiments, an Fc fragment variant comprises an amino acid sequence that is at least 93% identical to SEQ ID NO: 3. In some embodiments, an Fc fragment variant comprises an amino acid sequence that is at least 94% identical to SEQ ID NO: 3. In some embodiments, an Fc fragment variant comprises an amino acid sequence that is at least 95% identical to SEQ ID NO: 3. In some embodiments, an Fc fragment variant comprises an amino acid sequence that is at least 96% identical to SEQ ID NO: 3. In some embodiments, an Fc fragment variant comprises an amino acid sequence that is at least 97% identical to SEQ ID NO: 3. In some embodiments, an Fc fragment variant comprises an amino acid sequence that is at least 98% identical to SEQ ID NO: 3. In some embodiments, an Fc fragment variant comprises an amino acid sequence that is at least 99% identical to SEQ ID NO: 3. In some embodiments, an Fc fragment variant comprises an amino acid sequence that is 100% identical to SEQ ID NO: 3. In some embodiments, for each of the foregoing variants that have percent identity to SEQ ID NO: 3, the variant comprises the mutations of M252Y, S254T, T256E, M428L, H433K, and N434F.

In certain embodiments, the Fc fragment comprises a sequence that is at least 85%, 90%, 91%, 92%, 93%, 94%, 95%, 96%, 97%, 98%, or 99% identical to an amino acid sequence comprising SEQ ID NO: 4, provided that such fragment comprises the mutations of M252Y, S254T, T256E, M428L, H433K, and N434Y.

In some embodiments, an Fc fragment variant comprises an amino acid sequence that is at least 80% identical to SEQ ID NO: 4. In some embodiments, an Fc fragment variant comprises an amino acid sequence that is at least 85% identical to SEQ ID NO: 4. In some embodiments, an Fc fragment variant comprises an amino acid sequence that is at least 88% identical to SEQ ID NO: 4. In some embodiments, an Fc fragment variant comprises an amino acid sequence that is at least 90% identical to SEQ ID NO: 4. In some embodiments, an Fc fragment variant comprises an amino acid sequence that is at least 91% identical to SEQ ID NO: 4. In some embodiments, an Fc fragment variant comprises an amino acid sequence that is at least 92% identical to SEQ ID NO: 4. In some embodiments, an Fc fragment variant comprises an amino acid sequence that is at least 93% identical to SEQ ID NO: 4. In some embodiments, an Fc fragment variant comprises an amino acid sequence that is at least 94% identical to SEQ ID NO: 4. In some embodiments, an Fc fragment variant comprises an amino acid sequence that is at least 95% identical to SEQ ID NO: 4. In some embodiments, an Fc fragment variant comprises an amino acid sequence that is at least 96% identical to SEQ ID NO: 4. In some embodiments, an Fc fragment variant comprises an amino acid sequence that is at least 97% identical to SEQ ID NO: 4. In some embodiments, an Fc fragment variant comprises an amino acid sequence that is at least 98% identical to SEQ ID NO: 4. In some embodiments, an Fc fragment variant comprises an amino acid sequence that is at least 99% identical to SEQ ID NO: 4. In some embodiments, an Fc fragment variant comprises an amino acid sequence that is 100% identical to SEQ ID NO: 4. In some embodiments, for each of the foregoing variants that have percent identity to SEQ ID NO: 4, the variant comprises the mutations of M252Y, S254T, T256E, M428L, H433K, and N434Y.

In certain embodiments, the Fc fragment comprises a sequence that is at least 85%, 90%, 91%, 92%, 93%, 94%, 95%, 96%, 97%, 98%, or 99% identical to an amino acid sequence comprising SEQ ID NO: 5, provided that such fragment comprises the mutations of M252Y, S254T, T256E, H433K, and N434Y.

In some embodiments, an Fc fragment variant comprises an amino acid sequence that is at least 80% identical to SEQ ID NO: 5. In some embodiments, an Fc fragment variant comprises an amino acid sequence that is at least 85% identical to SEQ ID NO: 5. In some embodiments, an Fc fragment variant comprises an amino acid sequence that is at least 88% identical to SEQ ID NO: 5. In some embodiments, an Fc fragment variant comprises an amino acid sequence that is at least 90% identical to SEQ ID NO5 3. In some embodiments, an Fc fragment variant comprises an amino acid sequence that is at least 91% identical to SEQ ID NO: 5. In some embodiments, an Fc fragment variant comprises an amino acid sequence that is at least 92% identical to SEQ ID NO: 5. In some embodiments, an Fc fragment variant comprises an amino acid sequence that is at least 93% identical to SEQ ID NO: 5. In some embodiments, an Fc fragment variant comprises an amino acid sequence that is at least 94% identical to SEQ ID NO: 5. In some embodiments, an Fc fragment variant comprises an amino acid sequence that is at least 95% identical to SEQ ID NO: 5. In some embodiments, an Fc fragment variant comprises an amino acid sequence that is at least 96% identical to SEQ ID NO: 5. In some embodiments, an Fc fragment variant comprises an amino acid sequence that is at least 97% identical to SEQ ID NO: 5. In some embodiments, an Fc fragment variant comprises an amino acid sequence that is at least 98% identical to SEQ ID NO: 5. In some embodiments, an Fc fragment variant comprises an amino acid sequence that is at least 99% identical to SEQ ID NO: 5. In some embodiments, an Fc fragment variant comprises an amino acid sequence that is 100% identical to SEQ ID NO: 5. In some embodiments, for each of the foregoing variants that have percent identity to SEQ ID NO: 5, the variant comprises the mutations of M252Y, S254T, T256E, H433K, and N434Y.

In certain embodiments, the Fc fragment comprises a sequence that is at least 85%, 90%, 91%, 92%, 93%, 94%, 95%, 96%, 97%, 98%, or 99% identical to an amino acid sequence comprising SEQ ID NO: 6, provided that such fragment comprises the mutations of M252Y, S254T, T256E, M428L, and N434F.

In some embodiments, an Fc fragment variant comprises an amino acid sequence that is at least 80% identical to SEQ ID NO: 6. In some embodiments, an Fc fragment variant comprises an amino acid sequence that is at least 85% identical to SEQ ID NO: 36 In some embodiments, an Fc fragment variant comprises an amino acid sequence that is at least 88% identical to SEQ ID NO: 6. In some embodiments, an Fc fragment variant comprises an amino acid sequence that is at least 90% identical to SEQ ID NO: 6. In some embodiments, an Fc fragment variant comprises an amino acid sequence that is at least 91% identical to SEQ ID NO: 6. In some embodiments, an Fc fragment variant comprises an amino acid sequence that is at least 92% identical to SEQ ID NO: 6. In some embodiments, an Fc fragment variant comprises an amino acid sequence that is at least 93% identical to SEQ ID NO: 6. In some embodiments, an Fc fragment variant comprises an amino acid sequence that is at least 94% identical to SEQ ID NO: 6. In some embodiments, an Fc fragment variant comprises an amino acid sequence that is at least 95% identical to SEQ ID NO: 6. In some embodiments, an Fc fragment variant comprises an amino acid sequence that is at least 96% identical to SEQ ID NO: 6. In some embodiments, an Fc fragment variant comprises an amino acid sequence that is at least 97% identical to SEQ ID NO: 6. In some embodiments, an Fc fragment variant comprises an amino acid sequence that is at least 98% identical to SEQ ID NO: 6. In some embodiments, an Fc fragment variant comprises an amino acid sequence that is at least 99% identical to SEQ ID NO: 6. In some embodiments, an Fc fragment variant comprises an amino acid sequence that is 100% identical to SEQ ID NO: 6. In some embodiments, for each of the foregoing variants that have percent identity to SEQ ID NO: 6, the variant comprises the mutations of M252Y, S254T, T256E, M428L, and N434F.

In certain embodiments, the Fc fragment comprises a sequence that is at least 85%, 90%, 91%, 92%, 93%, 94%, 95%, 96%, 97%, 98%, or 99% identical to an amino acid sequence comprising SEQ ID NO: 7, provided that such fragment comprises the mutations of M252Y, S254T, T256E, H433R, and N434Y.

In some embodiments, an Fc fragment variant comprises an amino acid sequence that is at least 80% identical to SEQ ID NO: 7. In some embodiments, an Fc fragment variant comprises an amino acid sequence that is at least 85% identical to SEQ ID NO: 7. In some embodiments, an Fc fragment variant comprises an amino acid sequence that is at least 88% identical to SEQ ID NO: 7. In some embodiments, an Fc fragment variant comprises an amino acid sequence that is at least 90% identical to SEQ ID NO: 7. In some embodiments, an Fc fragment variant comprises an amino acid sequence that is at least 91% identical to SEQ ID NO: 7. In some embodiments, an Fc fragment variant comprises an amino acid sequence that is at least 92% identical to SEQ ID NO: 7. In some embodiments, an Fc fragment variant comprises an amino acid sequence that is at least 93% identical to SEQ ID NO: 7. In some embodiments, an Fc fragment variant comprises an amino acid sequence that is at least 94% identical to SEQ ID NO: 7. In some embodiments, an Fc fragment variant comprises an amino acid sequence that is at least 95% identical to SEQ ID NO: 7. In some embodiments, an Fc fragment variant comprises an amino acid sequence that is at least 96% identical to SEQ ID NO: 7. In some embodiments, an Fc fragment variant comprises an amino acid sequence that is at least 97% identical to SEQ ID NO: 7. In some embodiments, an Fc fragment variant comprises an amino acid sequence that is at least 98% identical to SEQ ID NO: 7. In some embodiments, an Fc fragment variant comprises an amino acid sequence that is at least 99% identical to SEQ ID NO: 7. In some embodiments, an Fc fragment variant comprises an amino acid sequence that is 100% identical to SEQ ID NO: 7. In some embodiments, for each of the foregoing variants that have percent identity to SEQ ID NO: 7, the variant comprises the mutations of M252Y, S254T, T256E, H433R, and N434Y.

In certain embodiments, the Fc fragment comprises a sequence that is at least 85%, 90%, 91%, 92%, 93%, 94%, 95%, 96%, 97%, 98%, or 99% identical to an amino acid sequence comprising SEQ ID NO: 8, provided that such fragment comprises the mutations of M252Y, S254T, T256E, H433R, and N434F.

In some embodiments, an Fc fragment variant comprises an amino acid sequence that is at least 80% identical to SEQ ID NO: 8. In some embodiments, an Fc fragment variant comprises an amino acid sequence that is at least 85% identical to SEQ ID NO: 8. In some embodiments, an Fc fragment variant comprises an amino acid sequence that is at least 88% identical to SEQ ID NO: 8. In some embodiments, an Fc fragment variant comprises an amino acid sequence that is at least 90% identical to SEQ ID NO: 8. In some embodiments, an Fc fragment variant comprises an amino acid sequence that is at least 91% identical to SEQ ID NO: 8. In some embodiments, an Fc fragment variant comprises an amino acid sequence that is at least 92% identical to SEQ ID NO: 8. In some embodiments, an Fc fragment variant comprises an amino acid sequence that is at least 93% identical to SEQ ID NO: 8. In some embodiments, an Fc fragment variant comprises an amino acid sequence that is at least 94% identical to SEQ ID NO: 8. In some embodiments, an Fc fragment variant comprises an amino acid sequence that is at least 95% identical to SEQ ID NO: 8. In some embodiments, an Fc fragment variant comprises an amino acid sequence that is at least 96% identical to SEQ ID NO: 8. In some embodiments, an Fc fragment variant comprises an amino acid sequence that is at least 97% identical to SEQ ID NO: 8. In some embodiments, an Fc fragment variant comprises an amino acid sequence that is at least 98% identical to SEQ ID NO: 8. In some embodiments, an Fc fragment variant comprises an amino acid sequence that is at least 99% identical to SEQ ID NO: 8. In some embodiments, an Fc fragment variant comprises an amino acid sequence that is 100% identical to SEQ ID NO: 8. In some embodiments, for each of the foregoing variants that have percent identity to SEQ ID NO: 8, the variant comprises the mutations of M252Y, S254T, T256E, H433R, and N434F.

In certain embodiments, the Fc fragment comprises a sequence that is at least 85%, 90%, 91%, 92%, 93%, 94%, 95%, 96%, 97%, 98%, or 99% identical to an amino acid sequence comprising SEQ ID NO: 9, provided that such fragment comprises the mutations of M252Y, S254T, T256E, M428L, H433R, and N434F.

In some embodiments, an Fc fragment variant comprises an amino acid sequence that is at least 80% identical to SEQ ID NO: 9. In some embodiments, an Fc fragment variant comprises an amino acid sequence that is at least 85% identical to SEQ ID NO: 9. In some embodiments, an Fc fragment variant comprises an amino acid sequence that is at least 88% identical to SEQ ID NO: 9. In some embodiments, an Fc fragment variant comprises an amino acid sequence that is at least 90% identical to SEQ ID NO: 9. In some embodiments, an Fc fragment variant comprises an amino acid sequence that is at least 91% identical to SEQ ID NO: 9. In some embodiments, an Fc fragment variant comprises an amino acid sequence that is at least 92% identical to SEQ ID NO: 9. In some embodiments, an Fc fragment variant comprises an amino acid sequence that is at least 93% identical to SEQ ID NO: 9. In some embodiments, an Fc fragment variant comprises an amino acid sequence that is at least 94% identical to SEQ ID NO: 9. In some embodiments, an Fc fragment variant comprises an amino acid sequence that is at least 95% identical to SEQ ID NO: 9. In some embodiments, an Fc fragment variant comprises an amino acid sequence that is at least 96% identical to SEQ ID NO: 9. In some embodiments, an Fc fragment variant comprises an amino acid sequence that is at least 97% identical to SEQ ID NO: 9. In some embodiments, an Fc fragment variant comprises an amino acid sequence that is at least 98% identical to SEQ ID NO: 9. In some embodiments, an Fc fragment variant comprises an amino acid sequence that is at least 99% identical to SEQ ID NO: 9. In some embodiments, an Fc fragment variant comprises an amino acid sequence that is 100% identical to SEQ ID NO: 9. In some embodiments, for each of the foregoing variants that have percent identity to SEQ ID NO: 9, the variant comprises the mutations of M252Y, S254T, T256E, M428L, H433R, and N434F.

In certain embodiments, the Fc fragment comprises a sequence that is at least 85%, 90%, 91%, 92%, 93%, 94%, 95%, 96%, 97%, 98%, or 99% identical to an amino acid sequence comprising SEQ ID NO: 18, provided that such fragment comprises the mutations of M252Y, S254T, T256E, L309D, H433K, and N434F.

In certain embodiments, the Fc fragment comprises a sequence that is at least 85%, 90%, 91%, 92%, 93%, 94%, 95%, 96%, 97%, 98%, or 99% identical to an amino acid sequence comprising SEQ ID NO: 19, provided that such fragment comprises the mutations of M252Y, S254T, T256E, L309D, H433K, and N434Y.

In certain embodiments, the Fc fragment comprises a sequence that is at least 85%, 90%, 91%, 92%, 93%, 94%, 95%, 96%, 97%, 98%, or 99% identical to an amino acid sequence comprising SEQ ID NO: 20, provided that such fragment comprises the mutations of M252Y, S254T, T256E, L309D, M428L, H433K, and N434F.

In certain embodiments, the Fc fragment comprises a sequence that is at least 85%, 90%, 91%, 92%, 93%, 94%, 95%, 96%, 97%, 98%, or 99% identical to an amino acid sequence comprising SEQ ID NO: 21, provided that such fragment comprises the mutations of M252Y, S254T, T256E, L309D, M428L, H433K, and N434Y.

In certain embodiments, the Fc fragment comprises a sequence that is at least 85%, 90%, 91%, 92%, 93%, 94%, 95%, 96%, 97%, 98%, or 99% identical to an amino acid sequence comprising SEQ ID NO: 22, provided that such fragment comprises the mutations of M252Y, S254T, T256E, L309D, Q311K, H433K, and N434F.

In certain embodiments, the Fc fragment comprises a sequence that is at least 85%, 90%, 91%, 92%, 93%, 94%, 95%, 96%, 97%, 98%, or 99% identical to an amino acid sequence comprising SEQ ID NO: 23, provided that such fragment comprises the mutations of M252Y, S254T, T256E, L309D, Q311K, H433K, and N434Y.

For sequence comparison, generally one sequence acts as a reference sequence, to which a second sequence is compared against. When using a sequence comparison algorithm, test and reference sequences may be entered into a computer, subsequent coordinates may be designated, if necessary, and sequence algorithm program parameters may be designated. Any suitable algorithm may be used, including but not limited to Smith-Waterman alignment algorithm, Viterbi, Bayesians, Hidden Markov and the like. Default program parameters can be used, or alternative parameters can be designated. The sequence comparison algorithm may then be used to calculate the percent sequence identities for the test sequences relative to the reference sequence, based on the program parameters. Any suitable algorithm may be used, whereby a percent identity is calculated. Some programs for example, calculate percent identity as the number of aligned positions that identical residues, divided by the total number of aligned positions.

Methods of alignment of sequences for comparison are well-known in the art. Optimal alignment of sequences for comparison can be conducted, e.g., by the local homology algorithm of Smith and Waterman, Adv. Appl. Math. 2:482 (1981), by the homology alignment algorithm of Needleman and Wunsch, J. Mol. Biol. 48:443 (1970), by the search for similarity method of Pearson and Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., Eds. 1995 supplement)), each of which are hereby incorporated by reference in its entirety. Exemplary computer software to determine identity between two sequences include, but are not limited to, GCG program package, Devereux, J., et al., Nucleic Acids Research, 12 (1), 387 (1984)), BLASTP, BLASTN, and FASTA Altschul, S. F. et al., J Molec. Biol., 215, 403 (1990)), each of which are hereby incorporated by reference in its entirety.

In some embodiments, the Fc fragment comprises additional amino acid residues at the N-terminus or the C-terminus. In some embodiments, the Fc fragments provided for herein comprises a C-terminal lysine residue. In some embodiments, the Fc fragment has an additional 1, 2, 3, 4 or 5 amino acid residues at the N-terminus and/or the C-terminus.

### Fc Region

Unless otherwise specified herein, numbering of amino acid residues in the Fc region or constant region is according to the EU numbering system, also called the EU index, as described in Kabat et al, Sequences of Proteins of Immunological Interest, 5th Ed. Public Health Service, National Institutes of Health, Bethesda, MD, 1991. An “Fc polypeptide” of a dimeric Fc as used herein refers to one of the two polypeptides forming the dimeric Fc domain, i.e. a polypeptide comprising C-terminal constant regions of an immunoglobulin heavy chain, capable of stable self-association. For example, an Fc polypeptide of a dimeric IgG Fc comprises an IgG CH2 and an IgG CH3 constant domain sequence. In certain aspects, the Fc fragments disclosed herein comprise the C-terminal 226 amino acids of the full human IgG Fc region. An Fc can be of the class IgG, and may be further divided into subclasses (isotypes), e.g., IgG1, IgG2, IgG3, and IgG4. In certain aspects, the Fc fragments described herein are of the IgG1, subclass. In certain aspects, the Fc fragments described herein are variants of the human IgG1 Fc region set forth in SEQ ID NO: 1. In certain aspects, the Fc fragments described herein are of the IgG2 subclass. In certain aspects, the Fc fragments described herein are of the IgG4, subclass.

The terms “Fc receptor” and “FcR” are used to describe a receptor that binds to the Fc region of an antibody. For example, an FcR can be a native sequence human FcR. Generally, an FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Immunoglobulins of other isotypes can also be bound by certain FcRs (see, e.g., Janeway et al., Immuno Biology: the immune system in health and disease, (Elsevier Science Ltd., NY) (4th ed., 1999)). The term also includes the neonatal receptor, FcRn, which is responsible for the transfer of maternal IgGs to the fetus (Guyer et al., J. Immunol. 117:587 (1976); and Kim et al., J. Immunol. 24:249 (1994)). The Fc fragments described herein selectively bind FcRn. In certain aspects, the Fc fragments selectively bind a mammalian FcRn, including cyno, rat and/or mouse FcRn.

Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain (reviewed in Daëron, Annu. Rev. Immunol. 15:203-234 (1997)). FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein.

Modifications in the CH2 domain can affect the binding of FcRs to the Fc. A number of amino acid modifications in the Fc region are known in the art for selectively altering the affinity of the Fc for different Fc gamma receptors.

Exemplary mutations that alter the binding of FcRs to the Fc are listed below:
S298A/E333A/K334A, S298A/E333A/K334A/K326A (Lu Y, Vernes J M, Chiang N, et al. J Immunol Methods. 2011 Feb. 28; 365 (1-2): 132-41);
F243L/R292P/Y300L/V305I/P396L, F243L/R292P/Y300L/L235V/P396L (Stavenhagen J B, Gorlatov S, Tuaillon N, et al. Cancer Res. 2007 Sep. 15; 67 (18): 8882-90; Nordstrom J L, Gorlatov S, Zhang W, et al. Breast Cancer Res. 2011 Nov. 30; 13 (6): R123);
F243L (Stewart R, Thom G, Levens M, et al. Protein Eng Des Sel. 2011 September; 24 (9): 671-8.), S298A/E333A/K334A (Shields R L, Namenuk A K, Hong K, et al. J Biol Chem. 2001 Mar. 2; 276 (9): 6591-604);
S239D/I332E/A330L, S239D/I332E (Lazar G A, Dang W, Karki S, et al. Proc Natl Acad Sci USA. 2006 Mar. 14; 103 (11): 4005-10);
S239D/S267E, S267E/L328F (Chu S Y, Vostiar I, Karki S, et al. Mol Immunol. 2008 September; 45 (15): 3926-33);
S239D/D265S/S298A/I332E, S239E/S298A/K326A/A327H, G237F/S298A/A330L/I 332E, S239D/I332E/S298A, S239D/K326E/A330L/I332E/S298A, G236A/S239D/D270L/I332E, S239E/S267E/H268D, L234F/S267E/N325L, G237F/V266L/S267D and other mutations listed in WO2011/120134 and WO2011/120135, herein incorporated by reference. Therapeutic Antibody Engineering (by William R. Strohl and Lila M. Strohl, Woodhead Publishing series in Biomedicine No 11, ISBN 1 907568 37 9, October 2012) lists mutations on page 283.

In certain embodiments, the Fc fragments disclosed herein are variants of (e.g., comprise one or more amino acid substitutions as compared to) SEQ ID NO:1, wherein the one or more substitutions result in a measurable K_{D} at pH 7.4 compared to an Fc fragment of SEQ ID NO: 1, which does not detectably bind FcRn at pH 7.4 without the one or more substitutions.

In certain embodiments, the one or more amino acid substitutions results in increased Fc fragment half-life at pH 6.0 compared to an Fc fragment comprising a wild-type Fc region.

### Binding

The affinity of a molecule X for its partner Y can be represented by the dissociation equilibrium constant (K_{D}). The kinetic components that contribute to the dissociation equilibrium constant are described in more detail below. Affinity can be measured by common methods known in the art, including those described herein, such as surface plasmon resonance (SPR) technology (e.g., BIACORE®) or biolayer interferometry (e.g., FORTEBIO®).

With regard to the binding of an Fc fragment to a target molecule, the terms “bind,” “specific binding,” “specifically binds to,” “specific for,” “selectively binds,” and “selective for” a particular target (e.g., a polypeptide target such as FcRn) means binding that is measurably different from a non-specific or non-selective interaction (e.g., with a non-target molecule). Specific binding can be measured, for example, by measuring binding to a target molecule (e.g., FcRn) and comparing it to binding to a non-target molecule. Specific binding can also be determined by competition with a control molecule that mimics the target molecule. In that case, specific binding is indicated if the binding of the Fc fragment to the target molecule is competitively inhibited by the control molecule. In some embodiments, the affinity of an Fc fragment for a non-target molecule is less than about 50% of the affinity for FcRn (i.e., the K_{D} for FcRn is two times lower than the K_{D} for a non-target). In some embodiments, the affinity of an Fc fragment for a non-target molecule is less than about 40% of the affinity for FcRn. In some embodiments, the affinity of an Fc fragment for a non-target molecule is less than about 30% of the affinity for FcRn. In some embodiments, the affinity of an Fc fragment for a non-target molecule is less than about 20% of the affinity for FcRn. In some embodiments, the affinity of an Fc fragment for a non-target molecule is less than about 10% of the affinity for FcRn. In some embodiments, the affinity of an Fc fragment for a non-target molecule is less than about 1% of the affinity for FcRn. In some embodiments, the affinity of an Fc fragment for a non-target molecule is less than about 0.1% of the affinity for FcRn.

When used herein in the context of a first Fc fragment and a second Fc fragment or a wild-type IgG (the “second molecule”), the term “competes with” or “cross-competes with” indicates that the first Fc fragment and the second molecule compete for binding to a target (e.g., FcRn). In one exemplary assay, FcRn is coated on a surface and contacted with a first Fc fragment, after which a second molecule is added. In another exemplary assay, a first Fc fragment is coated on a surface and contacted with FcRn, and then a second molecule is added. If the presence of the first Fc fragment reduces binding of the second molecule, in either assay, then the Fc fragment competes with the second molecule. The term “competes with” also includes combinations where first Fc fragment reduces binding of the second molecule, but where no competition is observed when the first Fc fragment and second molecule are added in the reverse order. However, in some embodiments, the first Fc fragment and the second molecule inhibit binding of each other to FcRn, regardless of the order in which they are added. In some embodiments, the first Fc fragment reduces binding of the second molecule to its receptor by at least 25%, at least 50%, at least 60%, at least 70%, at least 80%, at least 85%, at least 90%, at least 95%, or at least 99% as measured in a competitive binding assay. A skilled artisan can select the concentrations of the first Fc fragments and the second molecules used in the competition assays based on the affinities of the Fc fragment and the second molecule for FcRn. The assays described in this definition are illustrative, and a skilled artisan can utilize any suitable assay to determine if Fc fragments compete with each other. Suitable assays are described, for example, in Cox et al., “Immunoassay Methods,” in Assay Guidance Manual [Internet], Updated Dec. 24, 2014 (ncbi.nlm.nih.gov/books/NBK92434/; accessed Sep. 29, 2015); Silman et al., Cytometry, 2001, 44:30-37; and Finco et al., J. Pharm. Biomed. Anal., 2011, 54:351-358; each of which is incorporated by reference in its entirety.

A test (or first) Fc fragment competes with a second molecule (e.g., a reference Fc fragment) if an excess of a test Fc fragment (e.g., at least 2×, 5×, 10×, 20×, or 100×) inhibits or blocks binding of the second molecule by, e.g., at least 50%, 60%, 70%, 75%, 80%, 85%, 90%, 95%, or 99% as measured in a competitive binding assay. For example, a second, competing Fc fragment can be identified by its ability to compete for binding to FcRn with a first Fc fragment described herein. In certain instances, the second molecule can block or inhibit binding of the first Fc fragment by, e.g., at least 50%, 60%, 70%, 75%, 80%, 85%, 90%, 95%, or 99% as measured in a competitive binding assay. In certain instances, the second molecule can displace the first Fc fragment by greater than 50%, 60%, 70%, 75%, 80%, 85%, 90%, 95%, or 99%.

In certain embodiments, the Fc fragment binds an FcRn sequence set forth in SEQ ID NO: 10-17.

In certain embodiments, the Fc fragment binds to an FcRn sequence set forth in SEQ ID NO: 10-17 with a K_{D} of less than or equal to about 1, 2, 3, 4, 5, 6, 7, 8, 9×10⁻⁸ M at pH 6.0, as measured by surface plasmon resonance (SPR). In certain embodiments, the Fc fragment binds to human FcRn with a K_{D} of less than or equal to about 1×10⁻⁸ M at pH 6.0, as measured by surface plasmon resonance (SPR). In certain embodiments, the Fc fragment binds to an FcRn sequence set forth in SEQ ID NO: 10-17 with a K_{D} of less than or equal to about 1×10⁻⁹M at pH 6.0, as measured by surface plasmon resonance (SPR). I

In certain embodiments, the Fc fragment binds an FcRn with a lower K_{D} at pH 6.0 compared to an Fc fragment comprising a wild-type Fc region at the same pH.

In some embodiments, an Fc fragment provided herein binds FcRn at a pH of 6.0 with a K_{D} of less than or equal to about 1×10⁻⁸, 1.1×10⁻⁸, 1.2×10⁻⁸, 1.3×10⁻⁸, 1.4×10⁻⁸, 1.5×10⁻⁸, 1.6×10⁻⁸, 1.7×10⁻⁸, 1.8×10⁻⁸, 1.9×10⁻⁸, 1.95×10⁻⁸, 2×10⁻⁸, 2.5×10⁻⁸, 3×10⁻⁸, 3.5×10⁻⁸, 4×10⁻⁸, 4.5×10⁻⁸, 5×10⁻⁸, 6×10⁻⁸, 7×10⁻⁸, 8×10⁻⁸, 9×10⁻⁸, 1×10⁻⁹, 1.1×10⁻⁹, 1.2×10⁻⁹, 1.3×10⁻⁹, 1.4×10⁻⁹, 1.5×10⁻⁹, 1.6×10⁻⁹, 1.7×10⁻⁹, 1.8×10⁻⁹, 1.9×10⁻⁹, 1.95×10⁻⁹, 2×10⁻⁹, 2.5×10⁻⁹, 3×10⁻⁹, 3.5×10⁻⁹, 4×10⁻⁹, 4.5×10⁻⁹, 5×10⁻⁹, 6×10⁻⁹, 7×10⁻⁹, 8×10⁻⁹, 9×10⁻⁹, 1×10⁻¹⁰, 1.1×10⁻¹⁰, 1.2×10⁻¹⁰, 1.3×10⁻¹⁰, 1.4×10⁻¹⁰, 1.5×10⁻¹⁰, 1.6×10⁻¹⁰, 1.7×10⁻¹⁰, 1.8×10⁻¹⁰, 1.9×10⁻¹⁰, 1.95×10⁻¹⁰, 2×10⁻¹⁰, 2.5×10⁻¹⁰, 3×10⁻¹⁰, 3.5×10⁻¹⁰, 4×10⁻¹⁰, 4.5×10⁻¹⁰, 5×10⁻¹⁰, 6×10⁻¹⁰, 7×10⁻¹⁰, 8×10⁻¹⁰, or 9×10⁻¹⁰ M, as measured by ELISA or any other suitable method known in the art.

In some embodiments, the K_{D} of the Fc fragment provided herein for the binding of FcRn at a pH of 6.0 is from about 1.0-1.1×10⁻⁸ M, 1.1-1.2×10⁻⁸ M, 1.2-1.3×10⁻⁸ M, 1.3-1.4×10⁻⁸ M, 1.4-1.5×10⁻⁸ M, 1.5-1.6×10⁻⁸ M, 1.6-1.7×10⁻⁸ M, 1.7-1.8×10⁻⁸ M, 1.8-1.9×10⁻⁸ M, 1.9-2×10⁻⁸ M, 1-2×10⁻⁸ M, 1-5×10⁻⁸ M, 2-7×10⁻⁸ M, 3-8×10⁻⁸ M, 3-5×10⁻⁸ M, 4-6×10⁻⁸ M, 5-7×10⁻⁸ M, 6-8×10⁻⁸ M, 7-9×10⁻⁸ M, 7-9.9×10⁻⁸ M, 5-9.9×10⁻⁸ M, 1.0-1.1×10⁻⁹ M, 1.1-1.2×10⁻⁹ M, 1.2-1.3×10⁻⁹ M, 1.3-1.4×10⁻⁹ M, 1.4-1.5×10⁻⁹ M, 1.5-1.6×10⁻⁹ M, 1.6-1.7×10⁻⁹ M, 1.7-1.8×10⁻⁹ M, 1.8-1.9×10⁻⁹ M, 1.9-2×10⁻⁹ M, 1-2×10⁻⁹ M, 1-5×10⁻⁹ M, 2-7×10⁻⁹ M, 3-8×10⁻⁹ M, 3-5×10⁻⁹ M, 4-6×10⁻⁹ M, 5-7×10⁻⁹ M, 6-8×10⁻⁹ M, 7-9×10⁻⁹ M, 7-9.9×10⁻⁹ M, or 5-9.9×10⁻⁹ M as measured by ELISA or any other suitable method known in the art. In some embodiments, an Fc fragment provided herein binds FcRn at pH 6.0 with a K_{D} of less than or equal to about 1×10⁻⁸ M, or less than or equal to about 1×10⁻⁹ M as measured by ELISA or any other suitable method known in the art.

In some embodiments, an Fc fragment provided herein binds FcRn at a pH of 7.4 with a K_{D} of greater than or equal to about 1×10⁻⁵, 1.1×10⁻⁵, 1.2×10⁻⁵, 1.3×10⁻⁵, 1.4×10⁻⁵, 1.5×10⁻⁵, 1.6×10⁻⁵, 1.7×10⁻⁵, 1.8×10⁻⁵, 1.9×10⁻⁵, 1.95×10⁻⁵, 2×10⁻⁵, 2.5×10⁻⁵, 3×10⁻⁵, 3.5×10⁻⁵, 4×10⁻⁵, 4.5×10⁻⁵, 5×10⁻⁵, 6×10⁻⁵, 7×10⁻⁵, 8×10⁻⁵, 9×10⁻⁵, 1×10⁻⁶, 1.1×10⁻⁶, 1.2×10⁻⁶, 1.3×10⁻⁶, 1.4×10⁻⁶, 1.5×10⁻⁶, 1.6×10⁻⁶, 1.7×10⁻⁶, 1.8×10⁻⁶, 1.9×10⁻⁶, 1.95×10⁻⁶, 2×10⁻⁶, 2.5×10⁻⁶, 3×10⁻⁶, 3.5×10⁻⁶, 4×10⁻⁶, 4.5×10⁻⁶, 5×10⁻⁶, 6×10⁻⁶, 7×10⁻⁶, 8×10⁻⁶, 9×10⁻⁶, 1×10⁻⁷ 1.1×10⁻⁷, 1.2×10⁻⁷, 1.3×10⁻⁷, 1.4×10⁻⁷, 1.5×10⁻⁷, 1.6×10⁻⁷, 1.7×10⁻⁷, 1.8×10⁻⁷, 1.9×10⁻⁷, 1.95×10⁻⁷, 2×10⁻⁷, 2.5×10⁻⁷, 3×10⁻⁷, 3.5×10⁻⁷, 4×10⁻⁷, 4.5×10⁻⁷, 5×10⁻⁷, 6×10⁻⁷, 7×10⁻⁷, 8×10⁻⁷, 9×10⁻⁷, as measured by ELISA or any other suitable method known in the art.

In some embodiments, the K_{D} of the Fc fragment provided herein for the binding of FcRn at a pH of 6.0 is from about 1.0-1.1×10⁻⁵ M, 1.1-1.2×10⁻⁵ M, 1.2-1.3×10⁻⁵ M, 1.3-1.4×10⁻⁵ M, 1.4-1.5×10⁻⁵ M, 1.5-1.6×10⁻⁵ M, 1.6-1.7×10⁻⁵ M, 1.7-1.8×10⁻⁵ M, 1.8-1.9×10⁻⁵ M, 1.9-2×10⁻⁵ M, 1-2×10⁻⁵ M, 1-5×10⁻⁵ M, 2-7×10⁻⁵ M, 3-8×10⁻⁵ M, 3-5×10⁻⁵ M, 4-6×10⁻⁵ M, 5-7×10⁻⁵ M, 6-8×10⁻⁵ M, 7-9×10⁻⁵ M, 7-9.9×10⁻⁵ M, 5-9.9×10⁻⁵ M, 1.0-1.1×10⁻⁶ M, 1.1-1.2×10⁻⁶ M, 1.2-1.3×10⁻⁶ M, 1.3-1.4×10⁻⁶ M, 1.4-1.5×10⁻⁶ M, 1.5-1.6×10⁻⁶ M, 1.6-1.7×10⁻⁶ M, 1.7-1.8×10⁻⁶ M, 1.8-1.9×10⁻⁶ M, 1.9-2×10⁻⁶ M, 1-2×10⁻⁶ M, 1-5×10⁻⁶ M, 2-7×10⁻⁶ M, 3-8×10⁻⁶ M, 3-5×10⁻⁶M, 4-6×10⁻⁶ M, 5-7×10⁻⁶ M, 6-8×10⁻⁶ M, 7-9×10⁻⁶ M, 7-9.9×10⁻⁶ M, 5-9.9×10⁻⁶ M, 1.0-1.1×10⁻⁷ M, 1.1-1.2×10⁻⁷ M, 1.2-1.3×10⁻⁷ M, 1.3-1.4×10⁻⁷ M, 1.4-1.5×10⁻⁷ M, 1.5-1.6×10⁻⁷ M, 1.6-1.7×10⁻⁷ M, 1.7-1.8×10⁻⁷ M, 1.8-1.9×10⁻⁷ M, 1.9-2×10⁻⁷ M, 1-2×10⁻⁷ M, 1-5×10⁻⁷ M, 2-7×10⁻⁷ M, 3-8×10⁻⁷ M, 3-5×10⁻⁷ M, 4-6×10⁻⁷ M, 5-7×10⁻⁷ M, 6-8×10⁻⁷ M, 7-9×10⁻⁷ M, 7-9.9×10⁻⁷ M, 5-9.9×10⁻⁷ M, as measured by ELISA or any other suitable method known in the art. In some embodiments, an Fc fragment provided herein binds FcRn at pH 7.4 with a K_{D} of greater than or equal to about 1×10⁻⁵ M, or greater than or equal to about 1×10⁻⁶ M as measured by ELISA or any other suitable method known in the art.

In some embodiments, the Fc fragment binds to a FcRn sequence set forth in SEQ ID NO: 10 or 11 with a K_{D} of less than or equal to about 1×10⁻⁸ M, 2×10⁻⁸ M, 3×10⁻⁸ M, 4×10⁻⁸ M, 5×10⁻⁸ M, 6×10⁻⁸ M, 7×10⁻⁸ M, 8×10⁻⁸ M, 9×10⁻⁸ M, 1×10⁻⁹ M, 2×10⁻⁹ M, 3×10⁻⁹ M, 4×10⁻⁹ M, 5×10⁻⁹ M, 6×10⁻⁹ M, 7×10⁻⁹ M, 8×10⁻⁹ M, or 9×10⁻⁹ M, at pH 6.0 as measured by surface plasmon resonance (SPR). In some embodiments, the Fc fragment binds to a FcRn sequence set forth in SEQ ID NO: 12 or 13 with a K_{D} of less than or equal to about 1×10⁻⁸ M, 2×10⁻⁸ M, 3×10⁻⁸ M, 4×10⁻⁸ M, 5×10⁻⁸ M, 6×10⁻⁸ M, 7×10⁻⁸ M, 8×10⁻⁸ M, 9×10⁻⁸ M, at pH 6.0 as measured by surface plasmon resonance (SPR). In some embodiments, the Fc fragment binds to a FcRn sequence set forth in SEQ ID NO: 14 or 15 with a K_{D} of less than or equal to about 1×10⁻¹⁰ M, 2×10⁻¹⁰ M, 3×10⁻¹⁰ M, 4×10⁻¹⁰ M, 5×10⁻¹⁰ M, 6×10⁻¹⁰ M, 7×10⁻¹⁰ M, 8×10⁻¹⁰ M, or 9×10⁻¹⁰ M, at pH 6.0 as measured by surface plasmon resonance (SPR). In some embodiments, the Fc fragment binds to a FcRn sequence set forth in SEQ ID NO: 16 or 17 with a K_{D} of less than or equal to about 1×10⁻⁹M, 2×10⁻⁹ M, 3×10⁻⁹ M, 4×10⁻⁹ M, 5×10⁻⁹ M, 6×10⁻⁹ M, 7×10⁻⁹ M, 8×10⁻⁹M, or 9×10⁻⁹M, at pH 6.0 as measured by surface plasmon resonance (SPR).

In some embodiments, the Fc fragment binds to a FcRn sequence set forth in SEQ ID NO: 10 or 11 with a K_{D} of greater than or equal to about 1×10⁻⁷M, 2×10⁻⁷ M, 3×10⁻⁷M, 4×10⁻⁷ M, 5×10⁻⁷ M, 6×10⁻⁷ M, 7×10⁻⁷ M, 8×10⁻⁷ M, or 9×10⁻⁷ M at pH 7.4 as measured by surface plasmon resonance (SPR). In some embodiments, the Fc fragment binds to a FcRn sequence set forth in SEQ ID NO: 12 or 13 with a K_{D} of greater than or equal to about 1×10⁻⁷ M, 2×10⁻⁷ M, 3×10⁻⁷ M, 4×10⁻⁷ M, 5×10⁻⁷M, 6×10⁻⁷ M, 7×10⁻⁷ M, 8×10⁻⁷ M, 9×10⁻⁷ M, 1×10⁻⁶ M, 2×10⁻⁶ M, 3×10⁻⁶ M, 4×10⁻⁶ M, 5×10⁻⁶ M, 6×10⁻⁶M, 7×10⁻⁶ M, 8×10⁻⁶ M, or 9×10⁻⁶ M, at pH 7.4 as measured by surface plasmon resonance (SPR). In some embodiments, the Fc fragment binds to a FcRn sequence set forth in SEQ ID NO: 14 or 15 with a K_{D} of greater than or equal to about 1×10⁻⁹ M, 2×10⁻⁹ M, 3×10⁻⁹M, 4×10⁻⁹M, 5×10⁻⁹M, 6×10⁻⁹ M, 7×10⁻⁹ M, 8×10⁻⁹ M, or 9×10⁻⁹M, at pH 7.4 as measured by surface plasmon resonance (SPR). In some embodiments, the Fc fragment binds to a FcRn sequence set forth in SEQ ID NO: 16 or 17 with a K_{D} of greater than or equal to about 1×10⁻⁸M, 2×10⁻⁸ M, 3×10⁻⁸ M, 4×10⁻⁸ M, 5×10⁻⁸ M, 6×10⁻⁸ M, 7×10⁻⁸ M, 8×10⁻⁸ M, or 9×10⁻⁸ M, at pH 7.4 as measured by surface plasmon resonance (SPR).

### Fc Fragment Variant Fusion Proteins

In some embodiments, Fc fragment variants of the present invention are fused or complexed to various protein, peptides, and antibody fragments.

In some embodiments, an Fc fragment variant is fused or complexed to a Fab. In some embodiments, an Fc fragment variant is fused or complexed to a scFv. In some embodiments, an Fc fragment variant is fused or complexed to a sdAb. In some embodiments, an Fc fragment variant is fused or complexed to a VHH. In some embodiments, an Fc fragment variant is fused or complexed to a VNAR.

In some embodiments, an Fc fragment variant is fused or complexed to an albumin-binding domain. In some embodiments, an Fc fragment variant is fused or complexed to an albumin-binding Fab. In some embodiments, an Fc fragment variant is fused or complexed to an albumin-binding scFv. In some embodiments, an Fc fragment variant is fused or complexed to an albumin-binding sdAb. In some embodiments, an Fc fragment variant is fused or complexed to an albumin-binding VHH. In some embodiments, an Fc fragment variant is fused or complexed to an albumin-binding VNAR.

In some embodiments, an Fc fragment variant is fused or complexed to an albumin or its variants.

In some embodiments, an Fc fragment variant is fused or complexed to an HSA-binding domain. In some embodiments, an Fc fragment variant is fused or complexed to an HSA-binding Fab. In some embodiments, an Fc fragment variant is fused or complexed to an HSA-binding scFv. In some embodiments, an Fc fragment variant is fused or complexed to an HSA-binding sdAb. In some embodiments, an Fc fragment variant is fused or complexed to an HSA-binding VHH. In some embodiments, an Fc fragment variant is fused or complexed to an HSA-binding VNAR.

### Pharmaceutical Compositions

The present application provides compositions comprising the Fc fragments including pharmaceutical compositions comprising any one or more of the Fc fragments described herein with one or more pharmaceutically acceptable excipients. In some embodiments, the composition is sterile. The pharmaceutical compositions generally comprise an effective amount of an Fc fragment.

These compositions can comprise, in addition to one or more of the Fc fragments disclosed herein, a pharmaceutically acceptable excipient, carrier, buffer, stabilizer or other materials well known to those skilled in the art. Such materials should be non-toxic and should not interfere with the efficacy of the active ingredient. The precise nature of the carrier or other material can depend on the route of administration, e.g. oral, intravenous, cutaneous or subcutaneous, nasal, intramuscular, intraperitoneal routes.

Pharmaceutical compositions for oral administration can be in tablet, capsule, powder or liquid form. A tablet can include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol can be included.

For intravenous, cutaneous or subcutaneous injection, or injection at the site of affliction, the active ingredient may be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives can be included, as required.

A contemplated Fc fragment to be administered to an individual may be administered in a “therapeutically effective amount” or “prophylactically effective amount” (as the case can be, although prophylaxis can be considered therapy), this being sufficient to show benefit to the individual. The actual amount administered, and rate and time-course of administration, will depend on the nature and severity of protein aggregation disease being treated. Prescription of treatment, e.g. decisions on dosage etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980.

In certain embodiments, the pharmaceutical compositions described herein are formulated for intravenous injection.

A composition can be administered alone or in combination with other treatments, either simultaneously or sequentially dependent upon the condition to be treated.

### Methods

### Methods of Preparation

Fc fragments described herein can be produced using recombinant methods and compositions, e.g., as described in U.S. Pat. No. 4,816,567. In an embodiment, isolated nucleic acid encoding an Fc fragment described herein is provided. Such nucleic acid may encode an amino acid sequence comprising an Fc fragment that binds FcRn described herein. In a further embodiment, one or more vectors (e.g., expression vectors) comprising such nucleic acid are provided. In an embodiment, the nucleic acid is provided in a multicistronic vector. In a further embodiment, a host cell comprising such nucleic acid is provided. In one such embodiment, a host cell comprises (e.g., has been transformed with): a vector comprising a nucleic acid that encodes an amino acid sequence comprising an Fc fragment disclosed herein. In an embodiment, the host cell is eukaryotic, e.g. a Chinese Hamster Ovary (CHO) cell, or human embryonic kidney (HEK) cell, or lymphoid cell (e.g., Y0, NS0, Sp20 cell). In one embodiment, a method of making an Fc fragment is provided, wherein the method comprises culturing a host cell comprising nucleic acid encoding the Fc fragment, as provided above, under conditions suitable for expression of the Fc fragment, and optionally recovering the Fc fragment from the host cell (or host cell culture medium).

For recombinant production of the Fc fragment, nucleic acid encoding an Fc fragment, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the Fc fragment).

When an Fc fragment is recombinantly produced by the host cells, the protein in certain embodiments is present at about 30%, about 25%, about 20%, about 15%, about 10%, about 5%, about 4%, about 3%, about 2%, or about 1% or less of the dry weight of the cells. When the Fc fragment is recombinantly produced by the host cells, the protein, in certain embodiments, is present in the culture medium at about 5 g/L, about 4 g/L, about 3 g/L, about 2 g/L, about 1 g/L, about 750 mg/L, about 500 mg/L, about 250 mg/L, about 100 mg/L, about 50 mg/L, about 10 mg/L, or about 1 mg/L or less of the dry weight of the cells. In certain embodiments, “substantially purified” Fc fragment produced by the methods described herein, has a purity level of at least about 30%, at least about 35%, at least about 40%, at least about 45%, at least about 50%, at least about 55%, at least about 60%, at least about 65%, at least about 70%, specifically, a purity level of at least about 75%, 80%, 85%, and more specifically, a purity level of at least about 90%, a purity level of at least about 95%, a purity level of at least about 99% or greater as determined by appropriate methods such as SDS/PAGE analysis, RP-HPLC, SEC, and capillary electrophoresis.

Suitable host cells for cloning or expression of Fc fragment-encoding vectors include prokaryotic or eukaryotic cells described herein.

Recombinant host cells or host cells are cells that include an exogenous polynucleotide, regardless of the method used for insertion, for example, direct uptake, transduction, f-mating, or other methods known in the art to create recombinant host cells. The exogenous polynucleotide may be maintained as a nonintegrated vector, for example, a plasmid, or alternatively, may be integrated into the host genome. Host cells can include CHO, derivatives of CHO, NS0, Sp20, CV-1, VERO-76, HeLa, HepG2, Per.C6, or BHK.

For example, Fc fragments may be produced in bacteria, in particular when glycosylation and Fc effector function are not needed. For expression of Fc fragments and polypeptides in bacteria, see, e.g., U.S. Pat. Nos. 5,648,237, 5,789,199, and 5,840,523. (See also Charlton, Methods in Molecular Biology, Vol. 248 (B.K.C. Lo, ed., Humana Press, Totowa, N.J., 2003), pp. 245-254, describing expression of Fc fragments in E. coli.) After expression, the Fc fragment may be isolated from the bacterial cell paste in a soluble fraction and can be further purified.

In addition to prokaryotes, eukaryotic microbes such as filamentous fungi or yeast are suitable cloning or expression hosts for Fc fragment-encoding vectors, including fungi and yeast strains whose glycosylation pathways have been “humanized,” resulting in the production of a Fc fragment with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated Fc fragments are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as Y0, NS0 and Sp2/0. For a review of certain mammalian host cell lines suitable for a production, see, e.g., Yazaki and Wu, Methods in Molecular Biology, Vol. 248 (B.K.C. Lo, ed., Humana Press, Totowa, N.J.), pp. 255-268 (2003).

In an embodiment, the Fc fragments described herein are produced in stable mammalian cells, by a method comprising: transfecting at least one stable mammalian cell with: nucleic acid encoding the Fc fragment, in a predetermined ratio; and expressing the nucleic acid in the at least one mammalian cell. In some embodiments, the predetermined ratio of nucleic acid is determined in transient transfection experiments to determine the relative ratio of input nucleic acids that results in the highest percentage of the Fc fragment in the expressed product.

In some embodiments, is the method of producing a glycosylated Fc fragment in stable mammalian cells described herein, said method comprising identifying and purifying the desired glycosylated Fc fragment. In certain embodiments, the mammalian cell is selected and cultured under conditions for producing larger percentages of desired glycosylated Fc fragments. In some embodiments, the identification of the desired glycosylated Fc fragment is by one or both of liquid chromatography and mass spectrometry

If required, the Fc fragment can be purified or isolated after expression. Proteins may be isolated or purified in a variety of ways known to those skilled in the art. Standard purification methods include chromatographic techniques, including ion exchange, hydrophobic interaction, affinity, sizing or gel filtration, and reversed-phase, carried out at atmospheric pressure or at high pressure using systems such as FPLC and HPLC. Purification methods also include electrophoretic, immunological, precipitation, dialysis, and chromatofocusing techniques. Ultrafiltration and diafiltration techniques, in conjunction with protein concentration, are also useful. As is well known in the art, a variety of natural proteins bind Fc, and these proteins can find use in the present invention for purification of Fc fragments. For example, the bacterial proteins A and G bind to the Fc region. Purification can often be enabled by a particular fusion partner. For example, antibodies may be purified using glutathione resin if a GST fusion is employed, Ni⁺² affinity chromatography if a His-tag is employed or immobilized anti-flag antibody if a flag-tag is used. For general guidance in suitable purification techniques, see, e.g. incorporated entirely by reference Protein Purification: Principles and Practice, 3rd Ed., Scopes, Springer-Verlag, NY, 1994, incorporated entirely by reference.

In certain embodiments, the Fc fragment are purified using Anion Exchange Chromatography including, but not limited to, chromatography on Q-sepharose, DEAE sepharose, poros HQ, poros DEAF, Toyopearl Q, Toyopearl QAE, Toyopearl DEAE, Resource/Source Q and DEAE, Fractogel Q and DEAE columns.

In specific embodiments, the proteins described herein are purified using Cation Exchange Chromatography including, but not limited to, SP-sepharose, CM sepharose, poros HS, poros CM, Toyopearl SP, Toyopearl CM, Resource/Source S and CM, Fractogel S and CM columns and their equivalents and comparables.

In addition, Fc fragments described herein can be chemically synthesized using techniques known in the art (e.g., see Creighton, 1983, Proteins: Structures and Molecular Principles, W. H. Freeman & Co., N.Y and Hunkapiller et al., Nature, 310:105-111 (1984)). For example, a polypeptide corresponding to a fragment of a polypeptide can be synthesized by use of a peptide synthesizer. Furthermore, if desired, nonclassical amino acids or chemical amino acid analogs can be introduced as a substitution or addition into the polypeptide sequence. Nonclassical amino acids include, but are not limited to, to the D-isomers of the common amino acids, 2,4diaminobutyric acid, alpha-amino isobutyric acid, 4aminobutyric acid, Abu, 2-amino butyric acid, g-Abu, e-Ahx, 6amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, alanine, fluoro-amino acids, designer amino acids such as methyl amino acids, C-methyl amino acids, N-methyl amino acids, and amino acid analogs in general. Furthermore, the amino acid can be D (dextrorotary) or L (levorotary).

### Methods of Use

In an aspect, the present application provides methods of contacting FcRn with an Fc fragment as described herein, e.g., contacting a contemplated Fc fragment in-vivo or ex-vivo, which results in inhibition of IgG binding to the FcRn receptor. In some embodiments, the FcRn is expressed on a cell surface.

In an aspect, the present application provides methods of using the isolated Fc fragment described herein for treatment of a disorder or disease in a subject. In certain aspects, described herein is a method for treating a subject in need thereof with an Fc fragment as described herein, the method comprising administering to a mammalian subject a therapeutically effective amount of an Fc fragment or pharmaceutical composition comprising an Fc fragment described herein. In certain embodiments, the present application provides methods of treating a disorder or disease associated with elevated levels of IgG in a subject, by administering a disclosed Fc fragment.

In certain aspects, described herein are methods for treating a pathology associated with IgG activity, the method comprising administering to a mammalian subject a therapeutically effective amount an isolated Fc fragment or a pharmaceutical composition comprising an isolated Fc fragment described herein.

In certain aspects, described herein are methods for treating a pathology associated with elevated levels of FcRn in a mammalian subject in need thereof, the method comprising administering to the mammalian subject a therapeutically effective amount an Fc fragment or a pharmaceutical composition described herein.

In certain aspects, described herein is a method for treating or preventing a disorder or disease related to an antibody (e.g., an autoimmune disease or a disorder related to an unwanted side-effect of a therapeutic antibody) in a mammalian subject in need thereof, the method comprising administering to the mammalian subject a therapeutically effective amount the Fc fragment of any one of the embodiments disclosed herein or a pharmaceutical composition disclosed herein. In some embodiments, the inflammatory disorder or disease is an autoimmune disease. In some embodiments, the inflammatory disorder or disease is myasthenia gravis (gMG). In some embodiments, the inflammatory disorder or disease is immune thrombocytopenia (ITP).

In certain aspects, described herein is a method for treating a pathology associated with elevated levels of an IgG in a mammalian subject in need thereof, the method comprising administering to the mammalian subject a therapeutically effective amount the Fc fragment of any one of the embodiments disclosed herein or a pharmaceutical composition disclosed herein.

In certain aspects, described herein is a method of reducing biological activity of an IgG in a mammalian subject in need thereof, the method comprising administering to the mammalian subject a therapeutically effective amount the isolated Fc fragment of any one of the embodiments disclosed herein or a pharmaceutical composition disclosed herein.

In certain aspects, described herein is a method of treating or preventing autoimmune disease. In certain embodiments, the autoimmune disease is caused by auto-reactive antibodies.

In some embodiments, the autoimmune disease is selected from the group consisting of allogenic islet graft rejection, alopecia areata, ankylosing spondylitis, antiphospholipid syndrome, autoimmune Addison's disease, Alzheimer's disease, antineutrophil cytoplasmic autoantibodies (ANCA), autoimmune diseases of the adrenal gland, autoimmune hemolytic anemia, autoimmune hepatitis, autoimmune myocarditis, autoimmune neutropenia, autoimmune oophoritis and orchitis, autoimmune thrombocytopenia, autoimmune urticaria, Behcet's disease, bullous pemphigoid, cardiomyopathy, Castleman's syndrome, celiac spruce-dermatitis, chronic fatigue immune disfunction syndrome, chronic inflammatory demyelinating polyneuropathy (CIDP), Churg-Strauss syndrome, cicatrical pemphigoid, CREST syndrome, cold agglutinin disease, Crohn's disease, dermatomyositis, dilated cardiomyopathy, discoid lupus, epidermolysis bullosa acquisita, essential mixed cryoglobulinemia, factor VIII deficiency, fibromyalgia-fibromyositis, glomerulonephritis, Grave's disease, Guillain-Barre, Goodpasture's syndrome, graft-versus-host disease (GVHD), Hashimoto's thyroiditis, hemophilia A, idiopathic membranous neuropathy, idiopathic pulmonary fibrosis, idiopathic thrombocytopenia purpura (ITP), IgA neuropathy, IgM polyneuropathies, immune mediated thrombocytopenia, juvenile arthritis, Kawasaki's disease, lichen planus, lichen sclerosus, lupus erythematosus, Meniere's disease, mixed connective tissue disease, mucous membrane pemphigoid, multiple sclerosis, type 1 diabetes mellitus, Multifocal motor neuropathy (MMN), myasthenia gravis, paraneoplastic bullous pemphigoid, pemphigoid gestationis, pemphigus vulgaris, pemphigus foliaceus, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, relapsing polychondritis, Raynaud's phenomenon, Reiter's syndrome, rheumatoid arthritis, sarcoidosis, scleroderma, Sjogren's syndrome, solid organ transplant rejection, stiff-man syndrome, systemic lupus erythematosus, takayasu arteritis, toxic epidermal necrolysis (TEN), Stevens Johnson syndrome (SJS), temporal arteritis/giant cell arteritis, thrombotic thrombocytopenia purpura, ulcerative colitis, uveitis, dermatitis herpetiformis vasculitis, anti-neutrophil cytoplasmic antibody-associated vasculitides, vitiligo, and Wegner's granulomatosis.

### Methods of Administration and Manufacture

In some embodiments, the methods provided herein are useful for the treatment of a disease or disorder in an individual. In an embodiment, the individual is a human and the treatment comprises administration of an Fc fragment described herein.

In some embodiments, an Fc fragment is administered intravenously, intramuscularly, subcutaneously, topically, orally, transdermally, intraperitoneally, intraorbitally, by implantation, by inhalation, intrathecally, intraventricularly, or intranasally. In certain embodiments, an Fc fragment is administered intravenously. An effective amount of an Fc fragment may be administered for the treatment of a disease or disorder. The appropriate dosage of the Fc fragment may be determined based on the type of disease or disorder to be treated, the type of the Fc fragment, the severity and course of the disease or disorder, the clinical condition of the individual, the individual's clinical history and response to the treatment, and the discretion of the attending physician.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3^{rd} Ed. (Plenum Press) Vols A and B (1992).

### Methods

### Gene Synthesis and Plasmid Construction

The coding sequences for the Fc fragments were generated by DNA synthesis and PCR, subsequently subcloned into pcDNA3.1-based plasmid for protein expression in mammalian cell system. The gene sequences in the expression vectors were confirmed by DNA sequencing.

### Expression of Fc Fragments

Transient expression of antibodies was performed using the ExpiCHO™ Expression System (ThermoFisher, Cat. No. A29133). The ExpiCHO-S cells were expanded from a working cell bank and passaged in ExpiCHO expression media according to the manufacturer's instructions. The mixtures for transfection were prepared following the protocol instructions described in the ExpiCHO-S system manual. (Catalog Number A29133, Publication Number MAN0014337, Revision D.0). ExpiCHO cells were cultured for 14 days and the harvest was performed by filtration using 0.22 μm filtration units and DE as filter aid (Sartorius, Cat. No. SDLV-0150-05E0-2) after which they were processed immediately. The conditioned medium was harvested for protein purification.

### Purification of Fc Fragments

Produced antibodies were captured from clarified supernatants using a HiTrap MabSelect PrismA 25 mL column (Cytiva, Cat. nr. 17-5498-54) on an ÄKTA Pure 25 FPLC system. The column equilibration and protein binding were performed using 20 mM sodium phosphate with 150 mM NaCl pH 7.4 and for protein elution, it was used 100 mM sodium citrate with 150 mM NaCl pH 3.5. After elution, the peak corresponding to affinity purified antibodies was immediately neutralized with 30% of 1 M Tris pH 8.0. To polish the sample and to achieve a high purity >95% monomeric form, protein samples were loaded onto a HiLoad 26/600 Superdex 200 pg (Cytiva, Cat. nr. 28-9893-36) on an ÄKTA Pure 25 FPLC system. The fractions corresponding to the monomeric antibody form were pooled from a 96 deep well plate into a 50 ml falcon tube and filtered through a 0.22 μm PES membrane (Fisher brand, Cat. nr. 15206869, Lot nr. 2103171806) in a laminar flow chamber. Protein samples were transferred to 50 kDa MWCO spin concentrators (Amicon 50K Cat. nr. UFC905024; Lot nr. 0000187574) for concentration and each round of centrifugation was 10 min at 4000×g, and it was repeated until the desired concentration was achieved. SEC-HPLC analysis of Fc fragments

Analytical SEC-HPLC was performed using Thermo Vanquish Flex UHPLC system (Thermo Fisher) using a TSKgel Super SW mAb HTP (4.6 mm×15.0 cm) column (Tosoh Bioscience, Cat. nr. 00228559). Approximately 10 μg of sample was loaded. The mobile phase was 200 mM sodium phosphate, 0.05% sodium azide, pH 6.7 with a flow rate of 0.35 ml/min for 10 min, 25° C.

### Protein Thermal Stability Test by Differential Scanning Fluorimetry (DSF)

The Fc fragments in 20 mM sodium acetate pH 5.5 were loaded into a UNi (Unchained Labs). Samples were subjected to a thermal ramp from 20-95° C., with a ramp rate of 0.3° C./minute and ratio of the 330/350 nm fluorescence intensity and SLS at 266 nm against temperature were collected and UNCLE software was used to measure the peak of the 2nd derivatives of fluorescence ratio for the inflection points (Tm) of the transition curves, and SLS signal at 266 nm against temperature was used to determine Tagg.

### HIC-HPLC Analysis of Fc Fragments

HIC-HPLC was performed using a Proteomix HIC Butyl column with pre-column Proteomix HIC Butyl, on a UHPLC Vanquish Flex (ThermoFisher Scientific, MA), and using a salt gradient with a flow rate of 0.8 mL/min for 15 min. The detection was performed using a wavelength of 220 nm. The species eluted prior to the main peak, were assigned as lower hydrophobicity species, while the species eluted after the main peak was assigned as higher hydrophobicity species.

### Dynamic Light Scattering (DLS)

Protein size distribution and molecular size were monitored by dynamic light scattering (DLS) using UNCLE (Unchained Labs), 9 μL of each sample was loaded in triplicate into the UNI sample holder, and the intensity of scattered light was measured at 20° C., 10 acquisitions of 10 s each.

Non-Reducing and Reducing Capillary Electrophoresis Sodium Dodecyl Sulphate (nrCE-SDS and rCE-SDS)

CE-SDS was performed using LabChip (Perkin Elmer) or Maurice (ProteinSimple, CA). For CE-SDS using LabChip, approximately 2.5 μL of each sample were analysed using a Protein clear HT chip (cat nr CLS1486695) under non-reducing and reducing conditions, following the manufacturer's instructions. Reduced samples were treated with β-Mercaptoethanol and non-reduced samples were treated with iodoacetamide (IAM), prior to analysis. For CE-SDS using Maurice, 25 μg of each sample was analyzed using a CE-SDS cartridge (ProteinSimple, cat. nr. PS-MC02-SP) following manufacturers' instructions. Reduced samples were treated with β-Mercaptoethanol.

### Differential Scanning Calorimetry (DSC)

DSC experiments were carried out using a Microcal PEAQ DSC-DSC automated differential scanning microcalorimeter (Malvern Panalytical.). All solutions and samples used for DSC were filtered using a 0.22-μm filter and degassed prior to loading into the calorimeter. Antibodies used for the DSC studies were >95% monomeric as judged by analytical gel filtration chromatography. Prior to DSC analysis all samples were exhaustively dialyzed (at least three buffer exchanges) in 20 mM sodium acetate (pH 5.5). Buffer from this dialysis was then used as reference buffer for subsequent DSC experiments. Prior to sample measurement, baseline measurements (buffer-versus-buffer) were obtained to be subtracted from the sample measurement. 325 μL of each dialyzed sample (at a concentration of 0.5 mg/ml) was added to the sample well in duplicate and DSC measurements were performed at a 1.5° C./min scan rate. Data analysis and deconvolution were carried out using the DSC software provided by manufacturer.

### PEG Precipitation Assay

PEG precipitation assays were carried out similarly to Gibson et al., J. Pharm. Sci. 100:1009-1021 (2011). Addition of increasing amounts of PEG 4000 was used to precipitate the antibody. A 40% PEG stock solution in test buffer was prepared, from this, eight solutions, varying the final PEG percentage were prepared (ranging from 20% to 0%) in a 96-well plate by 1:1 dilution of protein samples in a total volume of 100 μL per well. The 96-well plate(s) were incubated overnight at room temperature, then read using a Microplate reader measuring the optical density at 320 nm.

Capillary Isoelectric Focusing (cIEF)

This work was conducted on a Maurice (ProteinSimple, CA) system, using a cIEF cartridge ProteinSimple, cat nr. PS-MC02-C), according to manufacturers' instructions. Prior to analysis, the samples were diluted in a master mix solution containing a mixture of ampholytes, pI markers, 1% methyl cellulose, 12.5% arginine and 4M urea. Example 1: Measuring Antibody Fragment-FcRn Binding Kinetics Using Surface Plasmon Resonance

A Biacore 8K SPR system (GE HealthCare) equipped with CM5 Sensor Chip (Cytiva, Cat. Nr. 29149603)) immobilized with an anti-human Fc specific antibody by amine coupling, was used to determine the binding kinetic rate and affinity constants at 25° C. and in a running buffer of 1×HBS-EP+pH 6.0 or pH 7.4 (10 mM HEPES, 150 mM NaCl, 3 mM EDTA, 0.05% Surfactant P20) (Cytiva, Cat. BR100669). Following a stabilization period in running buffer, the anti-FcRn mAb constructs at 20 nM were captured onto flow cell 2 (active) for 60 sec at a flow rate of 30 uL/min. Recombinant in-house Human FcRn protein was prepared at concentrations of 0, 26.25, 52.5, 125, 250, and 500 nM and injected over flow cell 1 (reference) and flow cell 2 (active) for 120 sec at a flow rate of 30 μL/min. Recombinant Cynomolgus FcRn protein, ((Immunitrack, Cat. ITF06-400)) was prepared at concentrations of 0, 25, 50, 100, 200, and 400 nM and injected over flow cell 1 (reference) and flow cell 2 (active) for 120 sec at a flow rate of 30 μL/min. Samples were injected in a multi-cycle manner over freshly captured mAb, by regenerating the capture surfaces with injection of 10 mM glycine pH 1.5 for 30 sec at a flow rate of 30 μL/min. The data was processed and analyzed with Biacore Insight Evaluation Software Version 2.0.15.12933 (GE Healthcare) as follows. Responses from flow cell 1 (reference) were subtracted from the responses from flow cell 2 (active). The responses from the two buffer blank injections were then subtracted from the reference subtracted data (2-1) to yield double-referenced data, which were fit to a 1:1 binding model to determine the apparent association (ka) and dissociation rate constants (kd). Their ratio provided the apparent equilibrium dissociation constant or affinity constant (KD=kd/ka).

TABLE 1
Binding Kinetics of FcRn from different species and Fc fragment variants at pH 6 and pH 7.4

| | HuFcRn KD (M) | | cynoFcRn KD (M) | | mouse_FcRn KD (M) | | rat_FcRn KD (M) | |
|---|---|---|---|---|---|---|---|---|
| mAb ID | pH 6.0 | pH 7.4 | pH 6.0 | pH 7.4 | pH 6 | pH 7.4 | pH 6 | pH 7.4 |
| Wt hIgG1 | 7.4E−7* | | 3.3E−7* | | 7.2E−8* | | 6.8E−8* | |
| Control | 8.80E−09 | 2.39E−07 | 3.97E−08 | 1.37E−06 | 2.69E−10 | 2.80E−09 | 3.71E−09 | 1.23E−08 |
| PAL02-0002 | | | | | | | | |
| Construct 1 | 8.05E−09 | 2.59E−07 | 3.62E−08 | 1.54E−06 | 1.82E−10 | 2.55E−09 | 3.50E−09 | 1.18E−08 |
| PAL02-0195 | | | | | | | | |
| Construct 2 | 6.62E−09 | 1.69E−07 | 2.86E−08 | 7.25E−07 | 1.43E−10 | 1.53E−09 | 3.49E−09 | 1.15E−08 |
| PAL02-0196 | | | | | | | | |
| Construct 3 | 6.74E−09 | 2.10E−07 | 2.89E−08 | 9.48E−07 | 1.72E−10 | 2.38E−09 | 4.25E−09 | 1.49E−08 |
| PAL02-0197 | | | | | | | | |
| Construct 4 | 8.41E−09 | 2.69E−07 | 3.86E−08 | 1.70E−06 | 2.48E−10 | 4.05E−09 | 2.90E−09 | 1.47E−08 |
| PAL02-0198 | | | | | | | | |
| Construct 5 | 7.15E−09 | 1.66E−07 | 3.01E−08 | 9.00E−07 | 1.94E−10 | 2.83E−09 | 5.41E−09 | 1.73E−08 |
| PAL02-0200 | | | | | | | | |
| Construct 6 | 9.53E−09 | 2.48E−07 | 4.17E−08 | 1.37E−06 | 2.00E−10 | 2.73E−09 | 4.33E−09 | 1.62E−08 |
| PAL02-0202 | | | | | | | | |
| Construct 7 | 8.26E−09 | 2.49E−07 | 3.61E−08 | 1.60E−06 | 2.88E−10 | 2.34E−09 | 3.94E−09 | 1.24E−08 |
| PAL02-0203 | | | | | | | | |

| | | | | | |
|---|---|---|---|---|---|
| PAL02-0204 | 7.64E−08 | 2.91E−07 | 6.08E−08 | 1.43E−06 | n.d. |
| PAL02-0205 | 5.31E−08 | 3.71E−07 | 4.72E−08 | 1.68E−06 | |
| PAL02-0206 | 6.83E−08 | 2.93E−07 | 6.21E−08 | 2.19E−06 | |
| PAL02-0207 | 5.87E−08 | 2.62E−07 | 5.12E−08 | 2.11E−06 | |
| PAL02-0208 | 8.04E−08 | 2.52E−07 | 1.25E−06 | 1.43E−06 | |
| PAL02-0209 | 5.17E−08 | 2.90E−07 | 3.72E−07 | 1.55E−06 | |

*kinetic values were adapted from a reference doi.org/10.1080/19420862.2015.1008353

### Example 2 Blocking IgG and PAL02-0002 Binding to Human FcRn by ELISA

The ability of the 7 constructs to block IgG and PAL02-0002, which is Efgartigimod without the C-terminal lysine, binding to human FcRn was measured by an ELISA assay. A competitive binding ELISA assay was used to screen for molecules that prevent IgG from binding to recombinant in-house human FcRn protein at pH 6.0. Candidate therapeutics prevent FcRn from binding IgG Abs in the acidic pH 6.0 endosomal environment. In this assay, functional huFcRn were coated at 2.0 μg/ml in 1×PBS to 96-well microtiter plates, and then used in an ELISA assay to determine the IC50 concentrations of test articles three-fold serial diluted starting at 100 nM down to 0.002 nM at pH 6.0 in the presence of 0.568 nM biotinylated human IgG1. Binding of biotinylated human IgG1 was revealed using ExtrAvidin-HRP (Sigma, Cat nr. E2886-1ML) at 1:5000 dilution. IC50 values were calculated using GraphPad Prism 7 software applying a nonlinear regression (curve fit) of a log (antagonist) vs. response-variable slop (four parameters). Fc Fragments that were therapeutic candidates were expected to efficiently inhibit the ability of the human IgG1 to bind FcRn. A similar procedure using biotinylated PAL02-0002 to replace biotinylated human IgG1 was performed for PAL02-0002 blocking.

As shown in Table 2, Constructs 1-7 were particularly effective in blocking IgG from binding to FcRn. Notably, constructs 1-6 had IC50 values that is half of EC50 value of Efigartigimod, and in particular, construct 3 had IC50 value of >3.5 fold less than that of Efigartigimod.

TABLE 2
Blocking IgG and PAL02-0002 Binding to human FcRn by ELISA

| | IC50 for IgG | IC50 for |
|---|---|---|
| | blocking in | Egartigimod |
| Construct | triplicate (nM) | inhibition (nM) |
| Wild-type Fc (PAL02-0001) | ND | ND |
| Control (PAL02-0002) | 2.85 | 4.82 |
| Construct 1 (PAL02-0195) | 1.31 | 4.09 |
| Construct 2 (PAL02-0196) | 1.48 | 5.03 |
| Construct 3 (PAL02-0197) | 0.77 | 4.59 |
| Construct 4 (PAL02-0198) | 1.51 | 6.47 |
| Construct 5 (PAL02-0200) | 1.41 | 3.73 |
| Construct 6 (PAL02-0202) | 1.13 | 4.71 |
| Construct 7 (PAL02-0203) | 2.09 | 3.85 |
| PAL02-0204 | 30.31 | n.d. |
| PAL02-0205 | 8.45 | |
| PAL02-0206 | 10.21 | |
| PAL02-0207 | 6.91 | |
| PAL02-0208 | 7.98 | |
| PAL02-0209 | 5.18 | |

### Example 3. Characterization of Fc Fragment Variants Binding FcRn

SEC-HPLC, HIC-HPLC analysis, and thermostability measurements of the FcRn constructs were performed as described in the Method section disclosed herein.

Dynamic light scattering (DLS), non-reducing capillary electrophoresis sodium dodecyl sulphate (nrCE-SDS), and reducing capillary electrophoresis sodium dodecyl sulphate (rCE-SDS) were performed as described in the Method section disclosed herein.

TABLE 3
Characterization of Fc fragment variants binding FcRn

| | | | | Hydro- | | | | | | | |
|---|---|---|---|---|---|---|---|---|---|---|---|
| | | Size | | phobicity | | | Size | | | | |
| | | SEC-HPLC | | HIC- | Thermostability | | DLS | | | NR CE- | R CE- |
| | | | Peak | HPLC | DSF/SLS | | | Mode | Peak | SDS | SDS |
| Sample | Titer | RT | width | RT | T_{M} | T_{AGG} | | Dia. | Mass | Mono- | Purity |
| ID | (mg/L) | (min) | (min) | (min) | (° C.) | (° C.) | PDI | (nm) | % | mer (%) | (%) |
| Wild- | 72.93 | 10.09 | 0.22 | 4.77 | 68.2 | 79.4 | 0.04 | 6.4 | 100 | 98.35 | 98.87 |
| type | | | | | | | | | | | |
| PAL02- | | | | | | | | | | | |
| 0001 | | | | | | | | | | | |
| Control | 155.64 | 10.07 | 0.21 | 7.97 | 59.4 | 76.9 | 0.03 | 6.6 | 100 | 98.77 | 98.37 |
| (PAL02- | | | | | | | | | | | |
| 0002) | | | | | | | | | | | |
| Construct 1 | 133.83 | 10.07 | 0.24 | 8.49 | 58.4 | 74.5 | 0.02 | 6.6 | 100 | 98.67 | 98.02 |
| (PAL02- | | | | | | | | | | | |
| 0195) | | | | | | | | | | | |
| Construct 2 | 130.83 | 9.92 | 0.26 | 8.77 | 58.9 | 74 | 0.06 | 7.4 | 100 | 98.54 | 98.17 |
| (PAL02- | | | | | | | | | | | |
| 0196) | | | | | | | | | | | |
| Construct 3 | 112.78 | 10.05 | 0.24 | 7.86 | 59.7 | 76.6 | 0.03 | 6.4 | 100 | 98.78 | 98.67 |
| (PAL02- | | | | | | | | | | | |
| 0197) | | | | | | | | | | | |
| Construct 4 | 71.43 | 9.99 | 0.22 | 9.08 | 57.8 | 70.9 | 0.08 | 6.6 | 100 | 98.82 | 99.6 |
| (PAL02- | | | | | | | | | | | |
| 0198) | | | | | | | | | | | |
| Construct 5 | 86.47 | 10.16 | 0.25 | 8.24 | 59.6 | 72.7 | 0.07 | 6.6 | 100 | 99.23 | 98.89 |
| (PAL02- | | | | | | | | | | | |
| 0200) | | | | | | | | | | | |
| Construct 6 | 168.42 | 10.09 | 0.22 | 8.38 | 59.2 | 74.7 | 0.04 | 6.7 | 100 | 98.88 | 98.76 |
| (PAL02- | | | | | | | | | | | |
| 0202) | | | | | | | | | | | |
| Construct 7 | 70.68 | 10.04 | 0.23 | 9.04 | 58.5 | 72.1 | 0.05 | 6.8 | 100 | 100 | 100 |
| (PAL02- | | | | | | | | | | | |
| 0203) | | | | | | | | | | | |
| Range | 70.7- | 9.9- | 0.2- | 4.8- | 57.8- | 70.9- | | 6.4- | | 98.4- | 98.0- |
| | 168.4 | 10.2 | 0.3 | 9.1 | 68.2 | 79.4 | | 7.4 | | 100.0 | 100.0 |

### Example 4. Summary of Fc Variants Developability Study

SEC-HPLC analysis was performed as described in the Method section disclosed herein.

Dynamic light scattering (DLS), differential scanning calorimetry (DSC), and PEG precipitation assay were performed as described in the Method section disclosed herein.

All samples showed a high monomer purity (>99%) before and after 3 freeze/thaw cycles. The DLS analysis showed the expected mode hydrodynamic diameter and low to intermediate polydispersity (PDI <0.2) for all samples. Only Wild-type (PAL02-0001) showed a TM value above 65° C. Most of the remaining samples showed a TM between 60-63° C.

TABLE 4
Characterization of FcRn constructs

| | | | | | | DLS | | | | | | | | | | PEG |
|---|---|---|---|---|---|---|---|---|---|---|---|---|---|---|---|---|
| | | | | | | | | Pk 1 | | | | | | | | precipitation |
| | | SEC-HPLC | | | | Z-Ave. | | Mode | | | | Pk 1 | | | | assay |
| | | Monomer | | HMWS | | Dia. | | Dia. | | | | Mass | | DSC | | PEG % |
| | Conc. | % | | % | | (nm) | | (nm) | | PDI | | (%) | | T_{M1} | T_{M2} | resulting in |
| | (mg/ | | 3 × | | 3 × | | 3 × | | 3 × | | 3 × | | 3 × | (° C.) | (° C.) | increased |
| Sample ID | ml) | T0 | FT | T0 | FT | T0 | FT | T0 | FT | T0 | FT | T0 | FT | CH2 | CH3 | A320 nm |
| Wild-type | 2.0 | 100.0 | 100.0 | 0.0 | 0.0 | 6.4 | 6.5 | 6.5 | 6.3 | 0.09 | 0.14 | 100.0 | 100.0 | 68.5 | 84.8 | 20 |
| PAL02- | | | | | | | | | | | | | | | | |
| 0001 | | | | | | | | | | | | | | | | |
| Control | 2.0 | 99.0 | 99.0 | 1.0 | 1.0 | 6.6 | 6.5 | 6.8 | 6.4 | 0.04 | 0.07 | 100.0 | 100.0 | 60.7 | 83.5 | 20 |
| (PAL02- | | | | | | | | | | | | | | | | |
| 0002) | | | | | | | | | | | | | | | | |
| Construct 1 | 2.0 | 99.1 | 99.1 | 0.9 | 0.9 | 6.6 | 6.6 | 6.8 | 6.4 | 0.04 | 0.08 | 100.0 | 100.0 | 60.0 | 80.8 | 18 |
| (PAL02- | | | | | | | | | | | | | | | | |
| 0195) | | | | | | | | | | | | | | | | |
| Construct 2 | 2.0 | 100.0 | 99.5 | 0.0 | 0.5 | 6.9 | 7.0 | 6.8 | 6.8 | 0.14 | 0.08 | 100.0 | 100.0 | 59.9 | 81.1 | 16 |
| (PAL02- | | | | | | | | | | | | | | | | |
| 0196) | | | | | | | | | | | | | | | | |
| Construct 3 | 2.0 | 99.5 | 99.5 | 0.5 | 0.5 | 6.5 | 6.2 | 6.5 | 6.3 | 0.07 | 0.06 | 100.0 | 100.0 | 60.6 | 83.8 | 18 |
| (PAL02- | | | | | | | | | | | | | | | | |
| 0197) | | | | | | | | | | | | | | | | |
| Construct 4 | 2.0 | 99.5 | 99.6 | 0.5 | 0.4 | 6.8 | 6.2 | 6.8 | 6.3 | 0.05 | 0.09 | 100.0 | 100.0 | 59.5 | 80.5 | 16 |
| (PAL02- | | | | | | | | | | | | | | | | |
| 0198) | | | | | | | | | | | | | | | | |
| Construct 5 | 2.0 | 100.0 | 100.0 | 0.0 | 0.0 | 6.6 | 6.4 | 6.7 | 6.0 | 0.06 | 0.15 | 100.0 | 100.0 | 60.5 | 83.3 | 16 |
| (PAL02- | | | | | | | | | | | | | | | | |
| 0200) | | | | | | | | | | | | | | | | |
| Construct 6 | 2.0 | 99.6 | 99.6 | 0.4 | 0.4 | 6.6 | 6.4 | 6.5 | 6.3 | 0.05 | 0.11 | 100.0 | 100.0 | 60.5 | 83.2 | 16 |
| (PAL02- | | | | | | | | | | | | | | | | |
| 0202) | | | | | | | | | | | | | | | | |
| Construct7 | 2.0 | 100.0 | 100.0 | 0.0 | 0.0 | 6.4 | 6.5 | 6.8 | 6.4 | 0.16 | 0.11 | 99.9 | 100.0 | 59.7 | 80.5 | 16 |
| (PAL02- | | | | | | | | | | | | | | | | |
| 0203) | | | | | | | | | | | | | | | | |

### Example 5. Short-Term Stability of Fc Variants

The short-term stability of Constructs 1-7 were tested in comparison to the wild-type Fc and PAL02-0002 at a concentration of 10 mg/ml, at 40° C., in 20 mM Sodium acetate pH 5.5 SEC-HPLC analysis was performed as described in the Method section disclosed herein. Dynamic light scattering (DLS), non-reducing capillary electrophoresis sodium dodecyl sulphate (nrCE-SDS), reducing capillary electrophoresis sodium dodecyl sulphate (nrCE-SDS), and capillary isoelectric focusing (cIEF) were performed as described in the Method section disclosed herein.

TABLE 5
Short-term stability of Fc Variants

| | | | | DLS | | | | cIEF | | | |
|---|---|---|---|---|---|---|---|---|---|---|---|
| | SEC- | | R CE- | | Pk 1 | | | | | | Main |
| | HPLC | NR CE- | SDS | Z-Ave. | Mode | Pk 1 | | Main | Acidic | Basic | peak |
| Sample ID | Mono- | SDS | Main | Dia. | Dia. | Mass | | peak | species | species | decrease |
| Monomer % | mer% | HC % | peak % | (nm) | (nm) | (%) | PDI | % | % | % | % |
| Wild type_T0 | 100 | 97.5 | 97.9 | 6.5 | 6.4 | 100 | 0.07 | 70 | 24.5 | 5.5 | −12.6 |
| Wild type_T3 days | 100 | 97.7 | 97.9 | 6.4 | 6.3 | 99.9 | 0.07 | 69.4 | 24.7 | 5.9 | |
| Wild type_T1 week | 100 | 97.4 | 97.7 | 6.4 | 6.5 | 100 | 0.03 | 66.8 | 26.6 | 6.6 | |
| Wild type_T2 weeks | 100 | 97.6 | 97.5 | 6.5 | 6.7 | 100 | 0.02 | 57.4 | 34 | 8.6 | |
| PAL02-0002_T0 | 99.22 | 98.2 | 97.5 | 7.2 | 7.2 | 100 | 0.05 | 69.5 | 23.6 | 6.9 | −13.3 |
| PAL02-0002_T3 days | 99.17 | 98.2 | 97.5 | 7.2 | 7.4 | 100 | 0.07 | 68.5 | 24.3 | 7.2 | |
| PAL02-0002_T1 week | 99.14 | 97.8 | 97.4 | 7.1 | 7.4 | 100 | 0.05 | 62.9 | 28 | 9.1 | |
| PAL02-0002_T2 weeks | 99.06 | 97.9 | 97.3 | 7.2 | 7.1 | 100 | 0.08 | 56.2 | 33.9 | 9.9 | |
| Construct 1 | 99.26 | 97.8 | 97.1 | 6.8 | 6.8 | 100 | 0.03 | 63.8 | 30.4 | 5.8 | −10.5 |
| PAL02-0195_T0 | | | | | | | | | | | |
| Construct 1 | 99.16 | 97.4 | 96.8 | 6.9 | 6.7 | 100 | 0.07 | 61.9 | 32 | 6.1 | |
| PAL02-0195_T3 days | | | | | | | | | | | |
| Construct 1 | 99.15 | 97.5 | 96.9 | 7 | 6.9 | 100 | 0.05 | 57.7 | 34 | 8.3 | |
| PAL02-0195_T1 week | | | | | | | | | | | |
| Construct 1 | 99.06 | 97 | 96.3 | 7.1 | 7.3 | 100 | 0.03 | 53.3 | 39.4 | 7.3 | |
| PAL02-0195_T2 weeks | | | | | | | | | | | |
| Construct 2 | 99.46 | 98.1 | 97.3 | 10.1 | 10.7 | 100 | 0.09 | 74.9 | 18.8 | 6.3 | −14.9 |
| PAL02-0196_T0 | | | | | | | | | | | |
| Construct 2 | 99.39 | 98 | 97.3 | 10.1 | 10.2 | 100 | 0.09 | 73.9 | 19 | 7.1 | |
| PAL02-0196_T3 days | | | | | | | | | | | |
| Construct 2 | 99.3 | 97.7 | 97.2 | 10.4 | 11.5 | 100 | 0.09 | 66.8 | 23.9 | 9.3 | |
| PAL02-0196_T1 week | | | | | | | | | | | |
| Construct 2 | 99.23 | 97.5 | 96.8 | 10.2 | 10.8 | 100 | 0.09 | 60 | 30.3 | 9.7 | |
| PAL02-0196_T2 weeks | | | | | | | | | | | |
| Construct 3 | 99.7 | 98 | 97.6 | 6.9 | 6.7 | 100 | 0.05 | 70.7 | 23.4 | 5.9 | −13.8 |
| PAL02-0197_T0 | | | | | | | | | | | |
| Construct 3 | 99.54 | 98 | 97.5 | 6.7 | 6.9 | 100 | 0.03 | 68 | 25.7 | 6.3 | |
| PAL02-0197_T3 days | | | | | | | | | | | |
| Construct 3 | 99.76 | 97.8 | 97.4 | 6.6 | 6.8 | 100 | 0.04 | 65.3 | 26.9 | 7.8 | |
| PAL02-0197_T1 week | | | | | | | | | | | |
| Construct 3 | 99.34 | 97.8 | 97.2 | 6.7 | 6.8 | 100 | 0.04 | 56.9 | 34.5 | 8.6 | |
| PAL02-0197_T2 weeks | | | | | | | | | | | |
| Construct 4 | 100 | 98.2 | 98.3 | 6.9 | 7.2 | 100 | 0.04 | 72.8 | 24.5 | 2.7 | −13.2 |
| PAL02-0198_T0 | | | | | | | | | | | |
| Construct 4 | 99.33 | 97.7 | 98.2 | 7 | 6.9 | 100 | 0.04 | 70.3 | 26.1 | 3.6 | |
| PAL02-0198_T3 days | | | | | | | | | | | |
| Construct 4 | 99.03 | 97.7 | 96.8 | 7 | 7.1 | 100 | 0.05 | 66.8 | 29.6 | 3.6 | |
| PAL02-0198_T1 week | | | | | | | | | | | |
| Construct 4 | 98.85 | 97.7 | 97.7 | 6.9 | 7.1 | 100 | 0.05 | 59.6 | 36.1 | 4.3 | |
| PAL02-0198_T2 weeks | | | | | | | | | | | |
| Construct 5 | 99.62 | 98.6 | 97.7 | 6.7 | 6.8 | 100 | 0.06 | 68.5 | 25.9 | 5.6 | −12 |
| PAL02-0200_T0 | | | | | | | | | | | |
| Construct 5 | 99.57 | 95.9 | 97.7 | 6.7 | 6.9 | 100 | 0.03 | 67.2 | 27.1 | 5.7 | |
| PAL02-0200_T3 days | | | | | | | | | | | |
| Construct 5 | 99.62 | 98.2 | 97.5 | 6.9 | 7.2 | 100 | 0.03 | 63.3 | 30.5 | 6.2 | |
| PAL02-0200_T1 week | | | | | | | | | | | |
| Construct 5 | 99.57 | 98.2 | 97.2 | 7 | 7.2 | 100 | 0.04 | 56.5 | 35.3 | 8.2 | |
| PAL02-0200_T2 weeks | | | | | | | | | | | |
| Construct 6 | 99.72 | 98.3 | 98 | 6.9 | 7 | 100 | 0.04 | 72.2 | 22.3 | 5.5 | −12.9 |
| PAL02-0202_T0 | | | | | | | | | | | |
| Construct 6 | 99.71 | 98 | 97.9 | 6.8 | 7.1 | 100 | 0.04 | 70.9 | 22.5 | 6.6 | |
| PAL02-0202_T3 days | | | | | | | | | | | |
| Construct 6 | 99.71 | 98.1 | 96.8 | 7 | 7 | 100 | 0.06 | 66.3 | 26.2 | 7.5 | |
| PAL02-0202_T1 week | | | | | | | | | | | |
| Construct 6 | 99.46 | 97.8 | 97.7 | 7.3 | 7.6 | 100 | 0.05 | 59.3 | 32 | 8.7 | |
| PAL02-0202_T2 weeks | | | | | | | | | | | |
| Construct 7 | 100 | 98.2 | 97.7 | 7.7 | 7.6 | 100 | 0.08 | 66.5 | 27.7 | 5.8 | −11.8 |
| PAL02-0203_T0 | | | | | | | | | | | |
| Construct 7 | 100 | 98.3 | 97.9 | 7.6 | 7.8 | 100 | 0.05 | 64.2 | 29.2 | 6.6 | |
| PAL02-0203_T3 days | | | | | | | | | | | |
| Construct 7 | 99.66 | 98.1 | 97.6 | 7.7 | 7.8 | 100 | 0.06 | 59.9 | 32.2 | 7.9 | |
| PAL02-0203_T1 week | | | | | | | | | | | |
| Construct 7 | 99.63 | 98 | 97.4 | 7.7 | 8 | 100 | 0.05 | 54.7 | 36.3 | 9 | |
| PAL02-0203_T2 weeks | | | | | | | | | | | |

INFORMAL SEQUENCE LISTING

| Identifier | Description | Amino Acid sequence |
|---|---|---|
| WT human IgG1 | Human | DKTHTCPPCPAPELLGGPSVFLFPPKPKDTLMISRTPEVTC |
| Fc | IGH1 (221- | VVVDVSHEDPEVKFNWYVDGVEVHNAKTKPREEQYNST |
| (PAL02-0001) | 446) | YRVVSVLTVLHQDWLNGKEYKCKVSNKALPAPIEKTISK |
| (SEQ ID NO: 1) | | AKGQPREPQVYTLPPSRDELTKNQVSLTCLVKGFYPSDIA |
| | | VEWESNGQPENNYKTTPPVLDSDGSFFLYSKLTVDKSRW |
| | | QQGNVFSCSVMHEALHNHYTQKSLSLSPG |
| WT human IgG2 | Human | VECPPCPAPPVAGPSVFLFPPKPKDTLMISRTPEVTCVVV |
| Fc | IGHG2 (222- | DVSHEDPEVQFNWYVDGVEVHNAKTKPREEQFNSTFRV |
| (SEQ ID NO: 24) | 446) | VSVLTVVHQDWLNGKEYKCKVSNKGLPAPIEKTISKTKG |
| | | QPREPQVYTLPPSREEMTKNQVSLTCLVKGFYPSDIAVE |
| | | WESNGQPENNYKTTPPMLDSDGSFFLYSKLTVDKSRWQ |
| | | QGNVFSCSVMHEALHNHYTQKSLSLSPG |
| WT human IgG4 | Human | PPCPSCPAPEFLGGPSVFLFPPKPKDTLMISRTPEVTCVVV |
| Fc | IGHG4 (224- | DVSQEDPEVQFNWYVDGVEVHNAKTKPREEQFNSTYRV |
| (SEQ ID NO: 25) | 446) | VSVLTVLHQDWLNGKEYKCKVSNKGLPSSIEKTISKAKG |
| | | QPREPQVYTLPPSQEEMTKNQVSLTCLVKGFYPSDIA VE |
| | | WESNGQPENNYKTTPPVLDSDGSFFLYSRLTVDKSRWQE |
| | | GNVFSCSVMHEALHNHYTQKSLSLSLG |
| Control | hIgG1_Fc_ | DKTHTCPPCPAPELLGGPSVFLFPPKPKDTLYITREPEVTC |
| PAL02-0002 | M252Y/S254T/ | VVVDVSHEDPEVKFNWYVDGVEVHNAKTKPREEQYNST |
| (SEQ ID NO: 2) | T256E/H433K/ | YRVVSVLTVLHQDWLNGKEYKCKVSNKALPAPIEKTISK |
| | N434F | AKGQPREPQVYTLPPSRDELTKNQVSLTCLVKGFYPSDIA |
| | | VEWESNGQPENNYKTTPPVLDSDGSFFLYSKLTVDKSRW |
| | | QQGNVFSCSVMHEALKFHYTQKSLSLSPG |
| Construct 1 | IgG1 | DKTHTCPPCPAPELLGGPSVFLFPPKPKDTLYITREPEVTC |
| PAL02-0195 | Fc_M252Y/ | VVVDVSHEDPEVKFNWYVDGVEVHNAKTKPREEQYNST |
| (SEQ ID NO: 3) | S254T/T256E/ | YRVVSVLTVLHQDWLNGKEYKCKVSNKALPAPIEKTISK |
| | M428L/H433K/ | AKGQPREPQVYTLPPSRDELTKNQVSLTCLVKGFYPSDIA |
| | N434F | VEWESNGQPENNYKTTPPVLDSDGSFFLYSKLTVDKSRW |
| | | QQGNVFSCSVLHEALKFHYTQKSLSLSPG |
| Construct 2 | IgG1 | DKTHTCPPCPAPELLGGPSVFLFPPKPKDTLYITREPEVTC |
| PAL02-0196 | Fc_M252Y/ | VVVDVSHEDPEVKFNWYVDGVEVHNAKTKPREEQYNST |
| (SEQ ID NO: 4) | S254T/T256E/ | YRVVSVLTVLHQDWLNGKEYKCKVSNKALPAPIEKTISK |
| | M428L/H433K/ | AKGQPREPQVYTLPPSRDELTKNQVSLTCLVKGFYPSDIA |
| | N434Y | VEWESNGQPENNYKTTPPVLDSDGSFFLYSKLTVDKSRW |
| | | QQGNVFSCSVLHEALKYHYTQKSLSLSPG |
| Construct 3 | IgG1 | DKTHTCPPCPAPELLGGPSVFLFPPKPKDTLYITREPEVTC |
| PAL02-0197 | Fc_M252Y/ | VVVDVSHEDPEVKFNWYVDGVEVHNAKTKPREEQYNST |
| (SEQ ID NO: 5) | S254T/T256E/ | YRVVSVLTVLHQDWLNGKEYKCKVSNKALPAPIEKTISK |
| | H433K/N434Y | AKGQPREPQVYTLPPSRDELTKNQVSLTCLVKGFYPSDIA |
| | | VEWESNGQPENNYKTTPPVLDSDGSFFLYSKLTVDKSRW |
| | | QQGNVFSCSVMHEALKYHYTQKSLSLSPG |
| Construct 4 | IgG1 | DKTHTCPPCPAPELLGGPSVFLFPPKPKDTLYITREPEVTC |
| PAL02-0198 | Fc_M252Y/ | VVVDVSHEDPEVKFNWYVDGVEVHNAKTKPREEQYNST |
| (SEQ ID NO: 6) | S254T/T256E/ | YRVVSVLTVLHQDWLNGKEYKCKVSNKALPAPIEKTISK |
| | M428L/N434F | AKGQPREPQVYTLPPSRDELTKNQVSLTCLVKGFYPSDIA |
| | | VEWESNGQPENNYKTTPPVLDSDGSFFLYSKLTVDKSRW |
| | | QQGNVFSCSVLHEALHFHYTQKSLSLSPG |
| Construct 5 | IgG1 | DKTHTCPPCPAPELLGGPSVFLFPPKPKDTLYITREPEVTC |
| PAL02-0200 | Fc_M252Y/ | VVVDVSHEDPEVKFNWYVDGVEVHNAKTKPREEQYNST |
| (SEQ ID NO: 7) | S254T/T256E/ | YRVVSVLTVLHQDWLNGKEYKCKVSNKALPAPIEKTISK |
| | H433R/N434Y | AKGQPREPQVYTLPPSRDELTKNQVSLTCLVKGFYPSDIA |
| | | VEWESNGQPENNYKTTPPVLDSDGSFFLYSKLTVDKSRW |
| | | QQGNVFSCSVMHEALRYHYTQKSLSLSPG |
| Construct 6 | IgG1 | DKTHTCPPCPAPELLGGPSVFLFPPKPKDTLYITREPEVTC |
| PAL02-0202 | Fc_M252Y/ | VVVDVSHEDPEVKFNWYVDGVEVHNAKTKPREEQYNST |
| (SEQ ID NO: 8) | S254T/T256E/ | YRVVSVLTVLHQDWLNGKEYKCKVSNKALPAPIEKTISK |
| | H433R/N434F | AKGQPREPQVYTLPPSRDELTKNQVSLTCLVKGFYPSDIA |
| | | VEWESNGQPENNYKTTPPVLDSDGSFFLYSKLTVDKSRW |
| | | QQGNVFSCSVMHEALRFHYTQKSLSLSPG |
| Construct 7 | IgG1 | DKTHTCPPCPAPELLGGPSVFLFPPKPKDTLYITREPEVTC |
| PAL02-0203 | Fc_M252Y/ | VVVDVSHEDPEVKFNWYVDGVEVHNAKTKPREEQYNST |
| (SEQ ID NO: 9) | S254T/T256E/ | YRVVSVLTVLHQDWLNGKEYKCKVSNKALPAPIEKTISK |
| | M428L/H433R/ | AKGQPREPQVYTLPPSRDELTKNQVSLTCLVKGFYPSDIA |
| | N434F | VEWESNGQPENNYKTTPPVLDSDGSFFLYSKLTVDKSRW |
| | | QQGNVFSCSVLHEALRFHYTQKSLSLSPG |
| PAL02-0204 | IgG1 | DKTHTCPPCPAPELLGGPSVFLFPPKPKDTLYITREPE |
| (SEQ ID NO: 18) | Fc_M252Y/ | VTCVVVDVSHEDPEVKFNWYVDGVEVHNAKTKPR |
| | S254T/T256E/ | EEQYNSTYRVVSVLTVDHQDWLNGKEYKCKVSNK |
| | L309D/H433K/ | ALPAPIEKTISKAKGQPREPQVYTLPPSRDELTKNQV |
| | N434F | SLTCLVKGFYPSDIAVEWESNGQPENNYKTTPPVLD |
| | | SDGSFFLYSKLTVDKSRWQQGNVFSCSVMHEALKF |
| | | HYTQKSLSLSPG |
| PAL02-0205 | IgG1 | DKTHTCPPCPAPELLGGPSVFLFPPKPKDTLYITREPE |
| (SEQ ID NO: 19) | Fc_M252Y/ | VTCVVVDVSHEDPEVKFNWYVDGVEVHNAKTKPR |
| | S254T/T256E/ | EEQYNSTYRVVSVLTVDHQDWLNGKEYKCKVSNK |
| | L309D/H433K/ | ALPAPIEKTISKAKGQPREPQVYTLPPSRDELTKNQV |
| | N434Y | SLTCLVKGFYPSDIAVEWESNGQPENNYKTTPPVLD |
| | | SDGSFFLYSKLTVDKSRWQQGNVFSCSVMHEALKY |
| | | HYTQKSLSLSPG |
| PAL02-0206 | IgG1 | DKTHTCPPCPAPELLGGPSVFLFPPKPKDTLYITREPE |
| (SEQ ID NO: 20) | Fc_M252Y/ | VTCVVVDVSHEDPEVKFNWYVDGVEVHNAKTKPR |
| | S254T/T256E/ | EEQYNSTYRVVSVLTVDHQDWLNGKEYKCKVSNK |
| | L309D/M428L/ | ALPAPIEKTISKAKGQPREPQVYTLPPSRDELTKNQV |
| | H433K/N434F | SLTCLVKGFYPSDIAVEWESNGQPENNYKTTPPVLD |
| | | SDGSFFLYSKLTVDKSRWQQGNVFSCSVLHEALKFH |
| | | YTQKSLSLSPG |
| PAL02-0207 | IgG1 | DKTHTCPPCPAPELLGGPSVFLFPPKPKDTLYITREPE |
| (SEQ ID NO: 21) | Fc_M252Y/ | VTCVVVDVSHEDPEVKFNWYVDGVEVHNAKTKPR |
| | S254T/T256E/ | EEQYNSTYRVVSVLTVDHQDWLNGKEYKCKVSNK |
| | L309D/M428L/ | ALPAPIEKTISKAKGQPREPQVYTLPPSRDELTKNQV |
| | H433K/N434Y | SLTCLVKGFYPSDIAVEWESNGQPENNYKTTPPVLD |
| | | SDGSFFLYSKLTVDKSRWQQGNVFSCSVLHEALKY |
| | | HYTQKSLSLSPG |
| PAL02-0208 | IgG1 | DKTHTCPPCPAPELLGGPSVFLFPPKPKDTLYITREPE |
| (SEQ ID NO: 22) | Fc_M252Y/ | VTCVVVDVSHEDPEVKFNWYVDGVEVHNAKTKPR |
| | S254T/T256E/ | EEQYNSTYRVVSVLTVDHKDWLNGKEYKCKVSNK |
| | L309D/Q311K/ | ALPAPIEKTISKAKGQPREPQVYTLPPSRDELTKNQV |
| | H433K/N434F | SLTCLVKGFYPSDIAVEWESNGQPENNYKTTPPVLD |
| | | SDGSFFLYSKLTVDKSRWQQGNVFSCSVMHEALKF |
| | | HYTQKSLSLSPG |
| PAL02-0209 | IgG1 | DKTHTCPPCPAPELLGGPSVFLFPPKPKDTLYITREPE |
| (SEQ ID NO: 23) | Fc_M252Y/ | VTCVVVDVSHEDPEVKFNWYVDGVEVHNAKTKPR |
| | S254T/T256E/ | EEQYNSTYRVVSVLTVDHKDWLNGKEYKCKVSNK |
| | L309D/Q311K/ | ALPAPIEKTISKAKGQPREPQVYTLPPSRDELTKNQV |
| | H433K/N434Y | SLTCLVKGFYPSDIAVEWESNGQPENNYKTTPPVLD |
| | | SDGSFFLYSKLTVDKSRWQQGNVFSCSVMHEALKY |
| | | HYTQKSLSLSPG |
| Antigen | P55899 | MGVPRPQPWALGLLLFLLPGSLGAESHLSLLYHLTA |
| Sequence | FCGRN | VSSPAPGTPAFWVSGWLGPQQYLSYNSLRGEAEPCG |
| (SEQ ID NO: 10) | HUMAN | AWVWENQVSWYWEKETTDLRIKEKLFLEAFKALG |
| | IgG receptor | GKGPYTLQGLLGCELGPDNTSVPTAKFALNGEEFM |
| | FcRn large | NFDLKQGTWGGDWPEALAISQRWQQQDKAANKEL |
| | subunit p51 | TFLLFSCPHRLREHLERGRGNLEWKEPPSMRLKARP |
| | | SSPGFSVLTCSAFSFYPPELQLRFLRNGLAAGTGQGD |
| | | FGPNSDGSFHASSSLTVKSGDEHHYCCIVQHAGLAQ |
| | | PLRVELESPAKSSVLVVGIVIGVLLLTAAAVGGALL |
| | | WRRMRSGLPAPWISLRGDDTGVLLPTPGEAQDADL |
| | | KDVNVIPATA |
| Antigen | P61769 | MSRSVALAVLALLSLSGLEAIQRTPKIQVYSRHPAEN |
| Sequence | B2MG_HUMAN | GKSNFLNCYVSGFHPSDIEVDLLKNGERIEKVEHSDL |
| (SEQ ID NO: 11) | Beta-2- | SFSKDWSFYLLYYTEFTPTEKDEYACRVNHVTLSQP |
| | microglobulin | KIVKWDRDM |
| Antigen | Q8SPV9 | MRVPRPQPWALGLLLFLLPGSLGAESHLSLLYHLTA |
| Sequence | FCGRN_MACFA | VSSPAPGTPAFWVSGWLGPQQYLSYDSLRGQAEPC |
| (SEQ ID NO: 12) | IgG | GAWVWENQVSWYWEKETTDLRIKEKLFLEAFKAL |
| | receptor | GGKGPYTLQGLLGCELSPDNTSVPTAKFALNGEEFM |
| | FcRn large | NFDLKQGTWGGDWPEALAISQRWQQQDKAANKEL |
| | subunit p51 | TFLLFSCPHRLREHLERGRGNLEWKEPPSMRLKARP |
| | | GNPGFSVLTCSAFSFYPPELQLRFLRNGMAAGTGQG |
| | | DFGPNSDGSFHASSSLTVKSGDEHHYCCIVQHAGLA |
| | | QPLRVELETPAKSSVLVVGIVIGVLLLTAAAVGGAL |
| | | LWRRMRSGLPAPWISLRGDDTGSLLPTPGEAQDADS |
| | | KDINVIPATA |
| Antigen | Q8SPW0 | MSPSVALAVLALLSLSGLEAIQRTPKIQVYSRHPPEN |
| Sequence | B2MG_MA | GKPNFLNCYVSGFHPSDIEVDLLKNGEKMGKVEHS |
| (SEQ ID NO: 13) | CFA Beta- | DLSFSKDWSFYLLYYTEFTPNEKDEYACRVNHVTLS |
| | 2- | GPRTVKWDRDM |
| | microglobulin | |
| Antigen | Q61559 | MGMPLPWALSLLLVLLPQTWGSETRPPLMYHLTAV |
| Sequence | FCGRN_MOUSE | SNPSTGLPSFWATGWLGPQQYLTYNSLRQEADPCG |
| (SEQ ID NO: 14) | IgG | AWMWENQVSWYWEKETTDLKSKEQLFLEALKTLE |
| | receptor | KILNGTYTLQGLLGCELASDNSSVPTAVFALNGEEF |
| | FcRn large | MKFNPRIGNWTGEWPETEIVANLWMKQPDAARKES |
| | subunit p51 | EFLLNSCPERLLGHLERGRRNLEWKEPPSMRLKARP |
| | | GNSGSSVLTCAAFSFYPPELKFRFLRNGLASGSGNCS |
| | | TGPNGDGSFHAWSLLEVKRGDEHHYQCQVEHEGLA |
| | | QPLTVDLDSSARSSVPVVGIVLGLLLVVVAIAGGVL |
| | | LWGRMRSGLPAPWLSLSGDDSGDLLPGGNLPPEAEP |
| | | QGANAFPATS |
| Antigen | P01887 | MARSVTLVFLVLVSLTGLYAIQKTPQIQVYSRHPPE |
| Sequence | B2MG_MOUSE | NGKPNILNCYVTQFHPPHIEIQMLKNGKKIPKVEMS |
| (SEQ ID NO: 15) | Beta-2- | DMSFSKDWSFYILAHTEFTPTETDTYACRVKHASMA |
| | microglobulin | EPKTVYWDRDM |
| Antigen | P13599.1 | MGMSQPGVLLSLLLVLLPQTWGAEPRLPLMYHLAA |
| Sequence | FCGRN_RAT | VSDLSTGLPSFWATGWLGAQQYLTYNNLRQEADPC |
| (SEQ ID NO: 16) | IgG | GAWIWENQVSWYWEKETTDLKSKEQLFLEAIRTLE |
| | receptor | NQINGTFTLQGLLGCELAPDNSSLPTAVFALNGEEF |
| | FcRn large | MRFNPRTGNWSGEWPETDIVGNLWMKQPEAARKE |
| | subunit p51 | SEFLLTSCPERLLGHLERGRQNLEWKEPPSMRLKAR |
| | | PGNSGSSVLTCAAFSFYPPELKFRFLRNGLASGSGNC |
| | | STGPNGDGSFHAWSLLEVKRGDEHHYQCQVEHEGL |
| | | AQPLTVDLDSPARSSVPVVGIILGLLLVVVAIAGGVL |
| | | LWNRMRSGLPAPWLSLSGDDSGDLLPGGNLPPEAEP |
| | | QGVNAFPATS |
| Antigen | P07151 | MARSVTVIFLVLVSLAVVLAIQKTPQIQVYSRHPPEN |
| Sequence | B2MG_RAT | GKPNFLNCYVSQFHPPQIEIELLKNGKKIPNIEMSDLS |
| (SEQ ID NO: 17) | Beta-2- | FSKDWSFYILAHTEFTPTETDVYACRVKHVILKEPK |
| | microglobulin | TVTWDRDM |
| Efgartigimod | hIgG1_Fc_ | DKTHTCPPCPAPELLGGPSVFLFPPKPKDTLYITREPEVTC |
| (SEQ ID NO: 26) | M252Y/S254T/ | VVVDVSHEDPEVKFNWYVDGVEVHNAKTKPREEQYNST |
| | T256E/H433K/ | YRVVSVLTVLHQDWLNGKEYKCKVSNKALPAPIEKTISK |
| | N434F | AKGQPREPQVYTLPPSRDELTKNQVSLTCLVKGFYPSDIA |
| | | VEWESNGQPENNYKTTPPVLDSDGSFFLYSKLTVDKSRW |
| | | QQGNVFSCSVMHEALKFHYTQKSLSLSPGK |

1. An Fc fragment that binds neonatal Fc receptor (FcRn), wherein the Fc fragment comprises an amino acid sequence at least 95% identical to SEQ ID NO: 1 and amino acid substitutions M428L and N434F, further wherein the Fc fragment does not comprise an amino acid substitution of L309D.

2. The Fc fragment of claim 1, wherein the Fc fragment further comprises amino acid substitution H433K as compared to SEQ ID NO: 1.

3. The Fc fragment of claim 2, wherein the Fc fragment further comprises amino acid substitutions M252Y, S254T, and T256E as compared to SEQ ID NO: 1.

4. The Fc fragment of claim 1, wherein the Fc fragment comprises an amino acid sequence at least 95% identical to SEQ ID NO: 3.

5. The Fc fragment of claim 1, wherein the Fc fragment comprises an amino acid sequence of SEQ ID NO: 3.

6. The Fc fragment of claim 1, wherein the Fc fragment is fused or complexed to a half-life extension domain.

7. The Fc fragment of claim 6, wherein the half-extension domain is an albumin, an albumin binding domain, or an HSA binding domain.

8. A pharmaceutical composition comprising the Fc fragment of claim 1.

9. An Fc fragment that binds neonatal Fc receptor (FcRn), wherein the Fc fragment comprises an amino acid sequence at least 95% identical to SEQ ID NO: 1 and amino acid substitution M428L, further wherein the Fc fragment does not comprise an amino acid substitution of L309D.

10. The Fc fragment of claim 9, wherein the Fc fragment further comprises amino acid substitution H433K as compared to SEQ ID NO: 1.

11. The Fc fragment of claim 10, wherein the Fc fragment further comprises amino acid substitutions M252Y, S254T, and T256E as compared to SEQ ID NO: 1.

12. The Fc fragment of claim 9, wherein the Fc fragment comprises an amino acid sequence at least 95% identical to SEQ ID NO: 3.

13. The Fc fragment of claim 9, wherein the Fc fragment comprises an amino acid sequence of SEQ ID NO: 3.

14. The Fc fragment of claim 9, wherein the Fc fragment is fused or complexed to a half-life extension domain.

15. The Fc fragment of claim 14, wherein the half-extension domain is an albumin, an albumin binding domain, or an HSA binding domain.

16. A pharmaceutical composition comprising the Fc fragment of claim 9.
